(19) United States
(12) Patent Application Publication
(10) Pub. No.: US 2006/0188519 A1
(43) Pub. Date: Aug. 24, 2006

(54) Peptides, antibodies, and methods for the diagnosis of SARS

(76) Inventors: To Cheung, Hong Kong (HK); Mun Yee Deborah Au, Hong Kong (HK); Wing Ip Anthony Lo, Laguna City (HK); Ka Fai To, Shatin (HK); Chui Shan Sammy Chow, Tuen Mun (HK); Tsz Yee Tracy Tam, Tuen Mun (HK); Chun Wu, San Diego, CA (US)

Correspondence Address: EAGLE IP LIMITED, UNIT 1201, 12/F KWAI HUNG HOLDINGS CENTRE, 89 KING'S ROAD, NORTH POINT, HONG KONG, CN

(21) Appl. No.: 11/152,520

(22) Filed: Jun. 13, 2005

Related U.S. Application Data
(60) Provisional application No. 60/579,333, filed on Jun. 14, 2004.

(51) Int. Cl. A61K 39/21; A61K 39/12

(52) U.S. Cl. 424/204.1; 424/188.1

(57) ABSTRACT

The present invention discloses 27 immunoreactive peptides and antibodies immunologically reactive with said peptides. Such peptides and antibodies can be used for diagnosis of SARS-CoV infection as well as for preparing vaccination against SARS-CoV or a closely related coronaviruses.

### RELATED APPLICATIONS

This application is a nonprovisional application which claims priority under 35 U.S.C. §119(e) to U.S. Provisional Patent Application No. 60/579,333, filed Jun. 14, 2004, the disclosure of which is incorporated herein by reference in its entirety.

### FIELD OF INVENTION

The invention relates to immunoreactive peptides derived from the SARS-CoV virus and antibodies to these peptides. The peptides and antibodies are useful for diagnostic kits and methods for diagnosing SARS-CoV infection in a host sample. The peptides and antibodies can also be used in vaccines for prophylactic or therapeutic treatment.

### INTRODUCTION

Severe acute respiratory syndrome (SARS) is a disease that threatens to become a global epidemic. SARS came to global attention in February 2003. Easily transmissible and capable of killing 4-15% of the people who contract it, SARS has spread rapidly from its place of origin in China, to most of the rest of the world. As of May 5, 2003, more than 6,000 patients from 29 countries have been diagnosed with the disease (C. Drosten and W. Preiser, Kamps-Hoffmann SARS Reference, May 12, 2003, hypertext transfer protocol (http) on the worldwide web at amedeo.com/sars/sars.htm).

The agent responsible for SARS is a new coronavirus, SARS Co-V. The Coronaviruses are the largest enveloped RNA viruses, with a RNA genome of 28-32 kb. Coronaviruses exhibit a broad host range, infecting mammalian and avian species. M. Peiris and his group in Canada were first to successfully sequence the SARS Co-V genome, later confirmed by the Centers for Disease Control. The genome includes 11 open reading frames that correspond to regions predicted to encode polypeptides including polymerase proteins (polymerase 1a and 1b), spike protein (S), small membrane protein (E), membrane protein (M), and nucleocapsid protein (N). The SARS Co-V genome comprises 29,727 nucleotides with an organization that is similar to that of other coronaviruses (hypertext transfer protocol (http) on the worldwide web at cdc.gov/ncidod/sars/sequence.html).

Molecular assays for SARS-CoV have been developed by different groups (Centers for Disease Control). For example, PCT Publication No. 04/018998 to the University of Maryland describes methods for quantifying a nucleotide sequence in a sample, including from a SARS virus. Other tests assess the levels of anti-SARS antibodies in host serum. IgM and IgG antibodies appear and change in level during the course of infection. Laboratory tests for SARS include an enzyme immunoassay (ELISA), immunofluorescence assay (IFA) in which SARS-CoV-infected cells are fixed and patient antibodies bind and are detected by immunofluorescent-labelled secondary antibodies, a neutralization test (NT) which assesses and quantifies the capacity of patient sera to neutralize SARS-CoV infectivity on cell culture, and reverse transcription polymerase chain reaction (RT-PCR) tests.

However, results of these tests are inconsistent in their specificity and can provide a high level of false positive results (Public Health Guidance for Community-Level Preparedness and Response to Severe Acute Respiratory Syndrome (SARS) Version 2, Supplement F, Centers for Disease Control). In fact, the CDC recommends that only high risk patients get tested because of the high rate of false positive results (hypertext transfer protocol (http) on the worldwide web at cdc.gov/ncidod/sars/guidance/F/app7.htm).

Therefore, there is a need for more sensitive diagnostic assays that can detect an individual and biological samples infected with SARS.

There is also a need for more sensitive and/or specific diagnostic assays that can detect, and in particular, that can detect SARS in a manner that reduces the level of false positive results.

It is therefore an object of the invention to provide improved reagents, kits, and methods for detection of a SARS infection in a host.

### SUMMARY OF INVENTION

The inventors have surprisingly discovered that hosts infected with SARS-CoV carry antibodies that are strongly reactive with certain peptides derived from the SARS-CoV virus genome. Peptides for detecting SARS and SARS-like coronaviruses are thus provided in one aspect of the present invention, as well as antibodies reactive to these peptides and diagnostic kits including these peptides and antibodies, as other aspects of the invention. These immunoreactive peptides and antibodies can be used for diagnosis of a SARS-CoV infection as well as for vaccination of hosts infected with or at risk for exposure to SARS-CoV or a closely related corronavirus.

In one embodiment, an immunoreactive peptide derived from a small membrane or envelope protein is provided. In another embodiment, an immunoreactive peptide derived from a spike protein is provided. In yet other embodiments, a peptide derived from a nucleocapsid protein or from a hypothetical protein is provided. The immunoreactive peptide can have an amino acid sequence of, for example, between about 10 and about 25 amino acids, or more typically between about 10 and about 20 or between about 12 and about 20 or 12, 13, 14, 15, 16, 17, 18, 19 or 20 amino acids in length.

The immunoreactive peptide can comprise, consist essentially of, or consist of an amino acid sequence, having at least 80% identity to an amino acid selected from the group consisting of: SRVKNLNSSEGVPDLLV (SEQ ID No. 1), MADNGTITVEELKQLLEQ (SEQ ID No. 2), KLNTDHAGSNDNIALLVQ (SEQ ID No. 3), NGTITVEELKQL (SEQ ID No. 4), DKYFKNHTSPDVDLGD (SEQ ID No. 5), PSSKRFQPFQQFGRD (SEQ ID No. 6), KYRYLRHGKLRPFERD (SEQ ID No. 7), DVVRDLPSGFNTLKPI (SEQ ID No. 8), DVQAPNYTQHTSSMRG (SEQ ID No. 9), CKFDEDDSEPVLKGVKLHYT (SEQ ID No. 10), CTTFDDVQAPNYTQHTSS (SEQ ID No. 11), GVLTPSSKRFQPFQQ (SEQ ID No. 12), RDVSDFTDSVRDPKTSEI (SEQ ID No. 13), RQLQNSMSGASADSTQA (SEQ ID No. 14), MSDNGPQSNQRSAPRIT (SEQ ID No. 15), TDNNQNGGRNGARPKQRRP (SEQ ID No. 16), PKQRRPQGLPNNTASWFT (SEQ ID No. 17), DDKDPQFKDNVILLNKHIDA (SEQ ID No. 18), KHIDAYKTFPPTEPKKDKKK (SEQ ID No. 19), KDKKKKTDEAQPLPQRQKKQ (SEQ ID No. 20), CAMDPIYDEPTTTTSVPL (SEQ ID No. 21), KVTAFQHQNSKKTTK (SEQ ID No. 22), KHKKVSTNLCTHSFRKKQVR (SEQ ID No. 23), TKKNYSELDDEEPMELDYP (SEQ ID No. 24), CPSGTYEGNSPFHPLADN (SEQ ID No. 25), EDAMGQGQNSADPKVYP (SEQ ID No. 26), QMTKLATTEELPDEF (SEQ ID No. 27).

In one embodiment, the immunoreactive peptide is or includes an amino acid sequence of at least one of SEQ ID Nos. 1-27. In another embodiment, the amino acid sequence of the immunoreactive peptide can be substantially homologous to a sequence selected from SEQ ID Nos. 1-27. For example, the amino acid sequence of the immunoreactive peptide can be at least 80% identical to a sequence from SEQ ID Nos. 1-27. In another embodiment, the amino acid sequence of the immunoreactive peptide is at least 85%, at least 90%, at least 95% or at least 98% or 99% identical to a sequence selected from SEQ ID Nos. 1-27. The 1, 2, 5, 10 or 15% variation of the sequence of the peptides may originate from substitution of conserved amino acids. Conserved amino acids generally refers to amino acids having similar side chains.

In one embodiment, the amino acid sequence of the immunoreactive peptide comprises an amino acid sequence having at least 80% homology with a sequence selected from SEQ ID Nos. 1-27. In another embodiment, the amino acid sequence of the immunoreactive peptide comprises an amino acid sequence selected from SEQ ID Nos. 1-27. In another embodiment, the amino acid sequence of the immunoreactive peptide consists essentially of an amino acid selected from SEQ ID Nos. 1-27. In yet another embodiment the amino acid sequence of the immunoreactive peptide consists of an amino acid selected from SEQ ID Nos. 1-27. The peptide can be conjugated to a carrier molecule. The carrier molecule can be bovine serum albumin (BSA) or keyhole lymphocyte hemocyanin (KLH).

The inventors have also discovered antibodies that are immunoreactive to the peptides of the invention. The antibodies can be immunoreactive to peptides derived from a small membrane or envelope protein, a spike protein, a nucleocapsid protein or a hypothetical protein. In one embodiment, an antibody can be reactive to a peptide comprising, consisting essentially of, or consisting of an amino acid sequence selected from at least one of SEQ ID Nos. 1-27. The antibody can also be reactive to a peptide including an amino acid sequence substantially homologous to a sequence selected from SEQ ID Nos. 1-27. For example, the antibody can be reactive to a peptide including an amino acid sequence at least 80% identical to a sequence from SEQ ID Nos. 1-27. The antibody can also be reactive to a peptide including an amino acid sequence at least 85%, at least 90%, at least 95% or at least 98% or 99% identical to a sequence selected from SEQ ID Nos. 1-27. In another embodiment, the antibody can be reactive to a peptide including an amino acid sequence comprising a sequence selected from SEQ ID Nos. 1-27. In another embodiment, the antibody can be reactive to a peptide including an amino acid sequence consisting essentially of a sequence selected from SEQ ID Nos. 1-27. In another embodiment, the antibody can be reactive to a peptide including an amino acid sequence consisting of a sequence selected from SEQ ID Nos. 1-27.

The antibody can be, for example, a polyclonal antibody or a monoclonal antibody. In one embodiment, the antibody is produced in a host by injection of an immunoreactive peptide with an amino acid sequence selected from SEQ ID Nos. 1-27. The antibody can be an Ig molecule, including an IgG or an IgM antibody. In one embodiment, the host is a mammal. In another embodiment, the host is a rabbit or a rodent. In yet another embodiment, the host is a human. The host can also be avian.

In another aspect, the invention also provides a diagnostic kit and method for recognizing a SARS-CoV infection in a host, a sample taken from a host, or in a laboratory culture or sample. The host sample can, for example, be serum or a tissue biopsy sample. Typically the host is a mammal, and more typically a human.

A diagnostic kit can comprise, consist essentially of, or consist of a peptide of the invention. The peptide can be linked to a carrier molecule or to a solid support. The solid support can be glass or fiber. In certain embodiments, mixtures of peptides of the invention are provided that can produce at least one signal upon binding of an antibody in a host or sample. Mixtures of peptides can include any described mixture, including, but not limited to, a mixture of SEQ ID Nos. 2, 6, 10 and 20 or a mixture of SEQ ID Nos. 2, 6, 20 and 21, or SEQ ID Nos. 2 and 21, or SEQ ID Nos. 2 and 20. In certain embodiments, a reagent antibody is provided in the diagnostic kit that is reactive against the host or sample species. The reagent antibody can be linked to an agent that produces a signal, such as a fluorescent molecule. In other embodiments, a second reagent or antibody is provided that can bind to the reagent antibody and elicit a signal.

A diagnostic kit for recognizing SARS-CoV infection in a host or a sample can also include at least one antibody of the invention. The kit can comprise an antibody linked to a carrier molecule. The antibody can also be linked to a solid support. The diagnostic kit can also comprise a molecule bound to the antibody that can elicit a signal when the antibody binds to a peptide or antigen. In another embodiment, the kit additionally comprises a signaling reagent that can provide a measurable signal when the antibody binds to a peptide or antigen. The signaling reagent can be a second antibody that is reactive to the antibody or antibodies of the invention. The second antibody can be linked to a carrier molecule or a solid support. The signaling reagent or second antibody can be linked to a molecule that elicits a signal upon binding of a peptide or antigen to the antibody of the invention. In another embodiment, the kit also includes peptides bound to the antibody of the invention. In certain embodiments, the kit includes a reagent that can produce a signal upon release of a peptide from the antibody.

A diagnostic test method is also provided in yet another aspect of the present invention. The method can include contacting a peptide of the invention with a sample and measuring formation of a peptide-antibody complex. In one embodiment, the peptide can be contacted with a sample from an infected host or sample. The peptide can be linked to a carrier molecule or a solid support. The formation of a peptide-antibody complex can elicit a measurable signal. The peptide can be linked to a compound that provides a measurable signal. In another embodiment, the method includes contacting the peptide-antibody complex with a reagent antibody reactive against the host or sample species that can provide a measurable signal upon binding to the complex. In other embodiments, a second reagent antibody can bind to the first reagent antibody and produce a signal. In another embodiment, an experimental antibody bound to the peptide is provided that can provide a measurable signal when the peptide is unbound by contact with a sample.

The method can also include contacting an antibody of the invention with a sample and measuring formation or detecting presence of an antigen-antibody complex. In one embodiment, the antibody can be contacted with a sample from an infected host or sample. The antibody can be linked to a carrier molecule or a solid support. The formation of a antigen-antibody complex can elicit a measurable signal. The antibody can be linked to a compound that provides a measurable signal. The method can also include contacting the antigen-antibody complex with a signaling reagent that can provide a measurable signal upon binding to the complex. The signaling reagent can be contacted with the antibody before, during, or after contact with the sample. The signaling reagent can be a second antibody that is reactive to the antibody, antibodies, and/or antigen/antibody complex of the invention. The signaling reagent or second antibody can be linked to a carrier molecule or a solid support. The second antibody can be linked to a molecule that can produce a signal upon binding of a peptide or antigen to the antibody of the invention.

In another embodiment, the method comprises, consists essentially of, or consists of binding a peptide including an amino acid sequence selected from at least one of SEQ ID Nos. 1-27 to the antibody before contact with the sample and detecting a signal produced when the peptide is released from the antibody. The release can occur by direct binding competition upon formation of the an antigen-antibody complex.

In another embodiment, a diagnostic kit for the diagnosis of the SARS-CoV virus infection is provided, which comprises, consists essentially of, or consists of an amino acid sequence that is at least 80% identical to a peptide selected from the group consisting of SEQ ID NOs. 1-27.

A diagnostic kit can contain, for example, an array of at least two peptides comprising, consisting essentially of, or consisting of, a sequence that is at least 80% homologous to sequences selected from SEQ ID NOs. 1-27.

In another embodiment, a diagnostic kit for diagnosing the presence of the SARS-CoV virus or fragments thereof comprising at least one antibody immunologically reactive with the SARS-CoV virus and capable of forming an antigen-antibody complex therewith and at least one peptide comprising, consisting essentially of, or consisting of an amino acid sequence that is at least 80% identical to a peptide selected from SEQ ID NOs. 1-27.

In another embodiment, a method for the determination of a SARS-CoV virus infection is provided, by providing a peptide comprising, consisting essentially of, or consisting of a peptide that is at least 80% identical to a peptide selected from SEQ ID NOs. 1-27, mixing a sample with the peptide, and detecting the presence of an antigen-antibody complex.

In yet another embodiment, a method for determination of the presence of SARS-CoV in a test sample is provided. The method can involve providing an antibody that is immunologically reactive with a peptide comprising, consisting essentially of, or consisting of an amino acid sequence that is at least 80% identical to a peptide selected from SEQ ID NOs. 1-27.

The invention also provides a method for increasing a host immune response against SARS comprising, consisting essentially of, or consisting of an amino acid sequence that is at least 80% identical to SEQ ID Nos. 1-27, or an antibody of the invention. A method of vaccinating a host is provided comprising treating the host with a peptide or antibody of the invention. In another embodiment, a method of reducing SARS-CoV infection in a host comprising treating the host with an antibody of the invention is provided. In yet another embodiment, a vaccine against SARS-CoV infection comprising, consisting essentially of, or consisting of a peptide at least 80% identical to an amino acid sequence selected from SEQ ID Nos. 1-27, or an antibody of the invention, with or without an adjuvant is provided.

### BRIEF DESCRIPTION OF THE FIGURES

FIG. 1a and b are diagrams depicting the location of the peptides of the invention with respect to the proteins in SARS CoV that they were derived from.

FIG. 2 is an image of a membraneous FAST slide with a positive and negative control, and peptide Ags18 (SEQ ID No. 1) dotted on it in duplicate, reacted with a SARS serum sample. The reaction was carried out using Alexa 546 anti-human IgG as reporter.

FIG. 3 is an image of a membraneous FAST slide with a positive and negative control, and peptides Ags16 (SEQ ID No. 3), Ags17 (SEQ ID No. 2), and BSA conjugated Ags17, dotted on it in duplicate, reacted with a SARS serum sample. The reaction was carried out using Alexa 546 anti-human IgG as reporter.

FIG. 4 is an image of a membraneous FAST slide with a positive and negative control, and peptides Ags5 (SEQ ID No. 6), Ags23 (SEQ ID No. 10), and KLH conjugated Ags23, dotted on it in duplicate, reacted with a SARS serum sample. The reaction was carried out using Alexa 546 anti-human IgG as reporter.

FIG. 5 is an image of a membraneous FAST slide with a positive and negative control, and peptides Ags12 (SEQ ID No. 14), Ags13 (SEQ ID No. 15), Ags27 (SEQ ID No. 17) KLH conjugated Ags28 (SEQ ID No. 18), KLH conjugated Ags29 (SEQ ID No. 19) and KLH conjugated Ags30 (SEQ ID No. 20), dotted on it in duplicate, reacted with a SARS serum sample. The reaction was carried out using Alexa 546 anti-human IgG as reporter.

FIG. 6 is an image of a membraneous FAST slide with a positive and negative control, and peptides Ags15 (SEQ ID No. 24) and Ags21 (SEQ ID No. 21), dotted on it in duplicate, reacted with a SARS serum sample. The reaction was carried out using Alexa 546 anti-human IgG as reporter.

FIG. 7 is an image of a membraneous FAST slide with a positive and negative control, and peptide mixtures 1 and 2 (see text), dotted on it in duplicate, reacted with a SARS serum sample. The reaction was carried out using Alexa 546 anti-human IgG as reporter.

FIG. 8a and 8b are images of a membraneous FAST slide with a positive and negative control and mixture 1 and peptide Ags17 (SEQ ID No. 2), comparing detection of SARS IgG and IgM in a recently infected serum sample. FIG. 8a is an image using Alexa 546 anti-human IgG as the reporter; FIG. 8b is an image using Alexa 546 anti-human IgM as the reporter.

FIG. 9 is an image of a CSS glass substrate slide with a positive and negative control, and peptide Ags17 (SEQ ID No. 2) and Ags21 (SEQ ID No. 21), dotted on it in duplicate, reacted with a SARS serum sample. The reaction was carried out using Alexa 546 anti-human IgG as reporter.

FIG. 10 is an image of a nitrocellulose membrane AE100 with a positive and negative control, and KLH conjugated peptide Ags17 (SEQ ID No. 2) and Ags30 (SEQ ID No. 20) and mixture 1, contacted with a SARS serum sample and imaged using colloidal gold conjugated anti-human IgG as reporter.

FIG. 11 is a graph of competition for binding sites on SARS antibody-coated microspheres between labeled SARS peptide Ags5 (SEQ ID No. 6) and SARS-infected serum samples.

FIG. 12 is an image of immunohistochemical studies of anti-peptide antibody SARS-AbS13a on SARS-CoV infected (+) and uninfected (−) cultures of LoVo (LOVO) and Vero E6 (VERO). Positive cytoplasmic signals are shown in brown colour. The nuclei are counter-stained blue by haematoxylin.

FIG. 13 is an image of immunohistochemical studies of anti-peptide antibody SARS-Abs13a on terminal ileum biopsy (A), autopsy lung sections (B) and autopsy terminal ileum sections (C). SARS-CoV infected cells (arrows) were demonstrated by the strong cytoplasmic signals. The sections are counter-stained with haematoxylin.

### DETAILED DESCRIPTION

### DEFINITIONS

“Array” refers to any means that allows for the detection of the presence of targeted biomarkers in parallel through the use of an orderly arrangement of probes affixed to a solid support. An array analysis can make use of common assay systems such as microplates or standard blotting membranes, and can be created by hand or can make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray are typically less than 300 microns in diameter and these arrays can contain thousands of spots. In addition, arrays can be automated via robotic means. A microarray can be fabricated by high-speed robotics, generally on glass but sometimes on nylon substrates. “Probe” refers to either the peptides of the invention that are comprising, consisting or consisting essentially of an amino acid sequence selected from SEQ ID Nos. 1-27 or the antibodies reactive to these peptides.

As used herein, the terminology “consisting essentially of” refers generally to a peptide or polypeptide which includes the amino acid sequence of the peptides of the present invention along with additional amino acids at the carboxyl and/or amino terminal ends and which maintains the activity of the peptides of the present invention provided herein.

In a preferred embodiment, the terminology “consisting essentially of” may also refer to peptides or polypeptides which have less than 20 amino acid residues in addition to the peptide according to the present invention. In a more preferred embodiment, the same terminology refers to a peptides with less than 15 amino acid residues in addition to the peptide according to the instant invention. In an even more preferred embodiment, the same terminology refers to a peptides with less than 10 amino acid residues in addition to the peptide according to the instant invention. In another preferred embodiment, the same terminology refers to peptides or polypeptides with less than 6 amino acids in addition to one of the peptide of the present invention. In another preferred embodiment, the same terminology refers to peptides or polypeptides with less than 4 amino acids in addition to one of the peptide of the present invention. In the most preferred embodiment, the same terminology refers to peptides or polypeptides with less than 2 amino acids in addition to one of the peptide of the present invention.

As used herein, “sample” or “clinical sample” relates to any sample obtained from a host for use in carrying out the procedures of the present invention. In one aspect, the host is suffering from a disease or syndrome that is at least partially caused by a virus. The host may also be an asymptomatic considered to be at risk of viral infection. The sample may be a cellular sample such as a tissue sample, for example of lung tissue obtained as a biopsy or post-mortem, a fluid sample, such as blood, saliva, sputum, urine, cerebrospinal fluid, or a swabbed sample obtained by swabbing a mucus membrane surface such as nasal surface, a pharyngeal surface, a buccal surface, and the like, or it may be obtained from an excretion such as feces, or it may be obtained from other bodily tissues or body fluids commonly used in diagnostic testing. The sample can also be obtained from cultured material. A sample can comprise a SARS-CoV virus or an antibody to a protein expressed by the SARS-CoV virus. A sample can also comprise an antibody reactive with the peptides of the invention or a peptide of the invention. “Negative control” sample is a sample that is known to be from a host not infected with a SARS-CoV virus or a sample of other material known not to contain a SARS-CoV immunoreactive peptide or antibody reactive to a SARS-CoV peptide or antigen. “Positive” control is a sample used to ensure accuracy by providing a peptide or antibody or sample from a host known to be infected by SARS-CoV that is designed to elicit a response in a kit or method of the invention.

A “host” can be any unicellular or multicellular organism. A host is typically a mammal or a bird, and is most typically a human. A host can also be a cell line derived from an organism.

A “sample” can be taken from a host and can include a tissue sample such as a biopsy, a bodily fluid sample, such as a blood or serum sample, or a sample of cell culture media. A sample is typically taken from a living organism or cell, but can also be derived from a cadaver. A sample can include a SARS-CoV viral antigen or viral gene. In addition, a sample can include an antibody to a SARS virus or an antibody reactive with a peptide of the invention. A “control sample” is known to not comprise a SARS antigen.

An “immunoreactive peptide” used herein can mean a peptide that shows either direct or indirect immunoreactivity. For example, peptide having SEQ ID No. 5 shows negative result in the immuno-reactive test in serum, but the antibody raised by said peptide in mice is reactive to SARS virus. Said peptide is considered an immunoreactive peptide according to the present invention.

In this specification, when related to the compounds, methods and kits of the invention, the term “peptide-antibody complex” can refer to the composite formed between the peptides of the invention and any antibody reactive with the peptides of the invention. The term “antigen-antibody complex” refers to the composite formed between an antibody of the invention and any protein or other antigen that is found in any sample.

Peptides

The inventors have discovered that hosts infected with SARS-CoV carry antibodies strongly reactive with certain peptides derived from putative sequences of the SARS-CoV genome. The peptides can be produced by any means, such as synthetically or by recombinant means, such as by transformation of a cell with a nucleic acid encoding a peptide of at least one of SEQ ID Nos. 1-27. The peptides can typically include immunoreactive epitopes derived from the sequence of SARS-CoV virus or hypothetical proteins of the SARS-CoV genome. Methods for synthesizing small peptides are well known in the art such as through solid phase peptide synthesis or protein biosynthesis in bacteria, plants, or eukaryotic cells.

The peptides of the invention can be modified, such as for example, by the incorporation of D-amino acids, ‘unnatural’ amino acids (such as b-alanine, diiodotyrosine, omithine, hydroxyproline, b-naphthylalanine, etc.), at the termini with substituents such as DNP, FITC, biotin, long chain alkanes, para-nitroanilides, AMC, chemical modification such as halogenation (i.e. 5′-fluoro-substitution) etc., on certain side chains as with the phosphorylation of serine, threonine or tyrosine, or sulphation of tyrosine, or by cyclisation via disulphide bond formation or by formation of cyclic amides, or can be phosphorylated or linked as chains.

The inventors have discovered that several SARS-CoV virus proteins, including small membrane/envelope, membrane, spike, nucleocapsid proteins, and hypothetical proteins Sars 3a, 7a/b and 9b, can be biomarkers for SARS-CoV infection diagnosis and screening. Either or both the antigenic peptides or their counterpart antibodies can be used for various diagnostic application. The peptides can be provided as synthetic peptides, conjugated forms of these peptides, or as whole or part of larger recombinant or native proteins.

In one embodiment, an immunoreactive peptide that can be derived from a small membrane or envelope protein is provided. In another embodiment, an immunoreactive peptide can be derived from a spike protein. In yet other embodiments, a peptide derived from a nucleocapsid protein or from a hypothetical protein is provided. In another embodiment, the immunoreactive peptide can include an amino acid sequence of, for example, between 10 and 25 amino acids, or, more typically, between 10 and 20 or between 12 and 20 or 12, 13, 14, 15, 16, 17, 18, 19 or 20 amino acids in length.

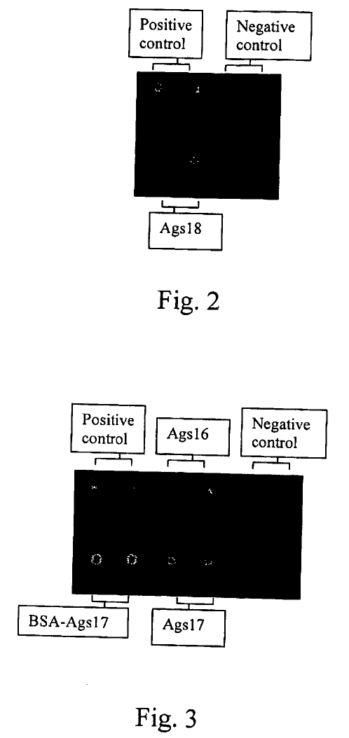

A peptide from the small membrane envelope protein, peptide SARS-AgS18 (SEQ ID No. 1) was found to react positively to SARS samples. About one out of 20 confirmed SARS cases appear to possess an antibody against a peptide including SEQ ID No. 1. No false positive cases were found in 100 normal individual. FIG. 2 shows the reaction of a peptide of SEQ ID No. 1 with a serum sample from a SARS-CoV infected host, using a fluorescently labeled, Alexa 546 anti-human IgG as a reporter. The reaction shown in FIG. 2 was carried out on a membraneous slide, termed “FAST” slide.

Peptides from membrane proteins SARS-AgS16 (SEQ ID No. 3), SARS-AgS17 (SEQ ID No. 2), and SARS-AgS31 (SEQ ID No. 4) were also found to react positively to SARS samples. About twelve out of 20 SARS patients have serum that reacts with a peptide including SEQ ID No. 3; and about 18 out of 20 SARS patients can have serum that reacts positively with a peptide including SEQ ID No. 2. Out of the 100 non-SARS samples, less than 5% were found to be false positive. FIG. 3 shows the reaction of a peptide including SEQ ID No. 3 and a peptide including SEQ ID No. 2 with a serum sample from a SARS-CoV infected host, using Alexa 546 anti-human IgG as reporter. The reaction depicted in FIG. 3 was carried out on membraneous FAST slide. A similar result was found for SEQ ID No. 4 but is not shown to avoid repetition.

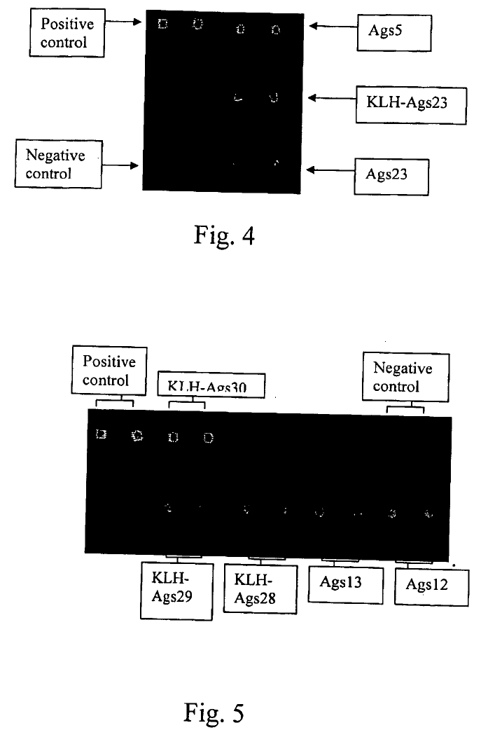

Spike Protein peptides SARS-AgS5 (SEQ ID No. 6), SARS-AgS23 (SEQ ID No. 10), SARS-AgS32 (SEQ ID No. 12), and SARS-AgS33 (SEQ ID No. 13) can also react positively with SARS samples. About 17 out of 20 samples from SARS infected hosts react positively with a peptide including SEQ ID No. 6; and about 11 out of 20 patients react positively with a peptide including SEQ ID No. 10. Out of the 100 non-SARS samples, less than 5% were found to be false positive. FIG. 4 shows the reaction of a peptide including SEQ ID No. 6 and a peptide including SEQ ID No. 10 with a serum sample from a SARS-CoV infected individual, using Alexa 546 anti-human IgG as reporter. Similar results were found for SEQ ID No. 12 and 13 but are not shown to avoid repetition.

Nucleocapsid Protein peptides SARS-AgS12 (SEQ ID No. 14), SARS-AgS13 (SEQ ID No. 15), SARS-AgS26 (SEQ ID No. 16), SARS-AgS27 (SEQ ID No. 17), SARS-AgS28 (SEQ ID No. 18), SARS-AgS29 (SEQ ID No. 19) and SARS-AgS30 (SEQ ID No. 20) can also react positively with SARS samples. Tests indicate that about 1 out of 20 SARS patient react positively with a peptide including SEQ ID No. 14; about 8 out of 12 SARS patient react positively with a peptide including SEQ ID No. 15, about 11 out of 20 for SARS patient react positively with a peptide including SEQ ID No. 17; about 5 out of 20 SARS patient react positively with a peptide including SEQ ID No. 18; about 18 out of 20 SARS patient react positively with a peptide including SEQ ID No. 19; and about 16 out of 20 SARS patient react positively with a peptide including SEQ ID No. 20. Out of the 100 non-SARS samples, less than about 5% were found to be false positive. FIG. 5 shows the reaction of peptides including SEQ ID No. 14, SEQ ID No. 15, SEQ ID No. 17, SEQ ID No. 18, SEQ ID No. 19 and SEQ ID No. 20 with a serum sample from a SARS-CoV infected host, using Alexa 546 anti-human IgG as reporter. A similar result was found for SEQ ID No. 16 but is not shown to avoid repetition.

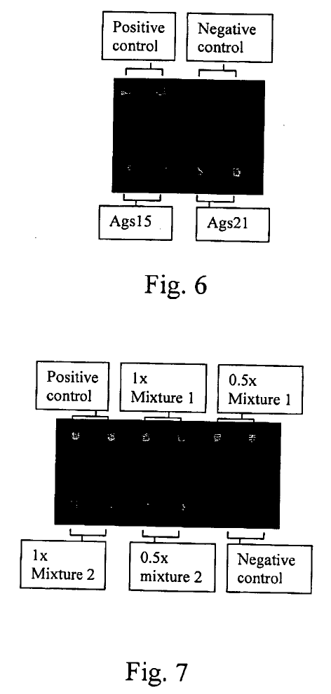

Several hypothetical proteins have been predicted from the sequencing of the SARS genome. Hypothetical proteins in SARS-CoV virus are putative and predicted by computer software and their existence has not been experimentally proven. However, the inventors have discovered that certain peptides derived from hypothetical SARS-CoV proteins can be immunoreactive with samples from SARS-infected host. Peptide SARS-AgS21 (SEQ ID No. 21), derived from hypothetical protein Sars 3a, can react positively with SARS samples. About 16 out of 20 samples from confirmed SARS infected hosts react positively with a peptide including SEQ ID No. 21. Out of the 100 non-SARS samples, less than 5% were found to be false positive. Peptide SARS-AgS19 (SEQ ID No. 23), derived from hypothetical protein Sars 3b, can react positively with SARS samples. About 16 out of 20 samples from confirmed SARS infected hosts react positively with a peptide including SEQ ID No. 23. Out of the 100 non-SARS samples, less than 5% were found to be false positive. Peptide SARS-AgS15 (SEQ ID No. 24), derived from hypothetical protein Sars 6, can react positively with SARS samples. About 11 out of 20 samples from confirmed SARS infected hosts react positively with a peptide comprising SEQ ID No. 24. Out of the 100 non-SARS samples, less than 5% were found to be false positive. Peptide SARS-AgS11 (SEQ ID No. 26), derived from hypothetical protein Sars 9b, also can react positively with SARS samples. About 1 out of 20 samples from confirmed SARS infected hosts react positively with a peptide including amino acids including SEQ ID No. 26. None out of 100 non-SARS samples were found false positive. FIG. 6 shows the reaction of a peptide including amino acids of SEQ ID No. 24 and SEQ ID No. 21 with a SARS serum sample, using Alexa 546 anti-human IgG as reporter. Similar results were found for SEQ ID No. 23 and 26 but are not shown to avoid repetition.

The immunoreactive peptide or peptides of the invention comprises, consists essentially of, or consists of an amino acid sequence selected from the following group: SRVKNLNSSEGVPDLLV (SEQ ID No. 1), MADNGTITVEELKQLLEQ (SEQ ID No. 2), KLNTDHAGSNDNIALLVQ (SEQ ID No. 3), NGTITVEELKQL (SEQ ID No. 4), DKYFKNHTSPDVDLGD (SEQ ID No. 5), PSSKRFQPFQQFGRD (SEQ ID No. 6), KYRYLRHGKLRPFERD (SEQ ID No. 7), DVVRDLPSGFNTLKPI (SEQ ID No. 8), DVQAPNYTQHTSSMRG (SEQ ID No. 9), CKFDEDDSEPVLKGVKLHYT (SEQ ID No. 10), CTTFDDVQAPNYTQHTSS (SEQ ID No. 11), GVLTPSSKRFQPFQQ (SEQ ID No. 12), RDVSDFTDSVRDPKTSEI (SEQ ID No. 13), RQLQNSMSGASADSTQA (SEQ ID No. 14), MSDNGPQSNQRSAPRIT (SEQ ID No. 15), TDNNQNGGRNGARPKQRRP (SEQ ID No. 16), PKQRRPQGLPNNTASWFT (SEQ ID No. 17), DDKDPQFKDNVILLNKHIDA (SEQ ID No. 18), KHIDAYKTFPPTEPKKDKKK (SEQ ID No. 19), KDKKKKTDEAQPLPQRQKKQ (SEQ ID No. 20), CAMDPIYDEPTTTTSVPL (SEQ ID No. 21), KVTAFQHQNSKKTTK (SEQ ID No. 22), KHKKVSTNLCTHSFRKKQVR (SEQ ID No. 23), TKKNYSELDDEEPMELDYP (SEQ ID No. 24), CPSGTYEGNSPFHPLADN (SEQ ID No. 25), EDAMGQGQNSADPKVYP (SEQ ID No. 26), QMTKLATTEELPDEF (SEQ ID No. 27).

In one embodiment, the immunoreactive peptide is or includes an amino acid sequence of at least one of SEQ ID Nos. 1-27. In another embodiment, the amino acid sequence of the immunoreactive peptide can be substantially homologous to a sequence selected from SEQ ID Nos. 1-27. For example, the amino acid sequence of the immunoreactive peptide is at least 80% identical to a sequence from SEQ ID Nos. 1-27. In another embodiment, the amino acid sequence of the immunoreactive peptide is at least 85%, at least 90%, at least 95% or at least 98% or 99% identical to a sequence selected from SEQ ID Nos. 1-27. The 1, 2, 5, 10 or 15% variation of the sequence of the peptides may originate from substitution of conserved amino acids. Conserved amino acids generally refers to amino acids having similar side chains. For example, a group of amino acids having basic side chains can be lysine, arginine, and histidine; a group of amino acids having acidic side chains can be aspartate and glutamate; a group of amino acids having aliphatic side chains can be glycine, alanine, valine, leucine, and isoleucine; a group of amino acids having aliphatic-hydroxyl side chains can be serine and threonine; a group of amino acids having uncharged hydrophilic side chains can be serine, threonine, asparagine and glutamine; a group of amino acids having inactive hydrophobic side chains can be glycine, alanine, valine, leucine and isoleucine; a group of amino acids having amide-containing side chains is asparagine and glutamine; a group of amino acids having aromatic side chains can be phenylalanine, tyrosine, and tryptophan; and a group of amino acids having sulfur-containing side chains can be cysteine and methionine.

In another embodiment the amino acid sequence of the immunoreactive peptide consists essentially of at least one of SEQ ID Nos. 1-27. In another embodiment the amino acid sequence of the immunoreactive peptide consists of at least one of SEQ ID Nos. 1-27. In one embodiment, the peptide is conjugated to a carrier molecule. In one embodiment, the carrier molecule can be bovine serum albumin (BSA) or keyhole lymphocyte hemocyanin (KLH).

The peptides of the invention can have the capacity to interact with antibodies reactive against the peptides. In particular, the peptides typically can have the capacity to form a peptide-antibody complex when contacted with a reactive antibody. Such reactive antibodies are typically found in SARS-infected hosts. In addition, recombinant reactive antibodies can be prepared as described herein.

A total of 33 peptides were synthesized and 27 were identified as immunoreactive. The sequences and the corresponding ID numbers of these immuno-active peptides and antibodies raised to the peptides are shown in Table 1.

TABLE 1
Summary of peptide sequence information and immunoreactivity of these peptides and their antibody counterparts.

| | | | | | | | | Immuno- |
|---|---|---|---|---|---|---|---|---|
| | | | | | | | | reactive |
| | | | | | Immuno- | | | antibody |
| | | | | | reactive | Immuno- | | for |
| | | | | | peptide | reactive | | immuno- |
| | | SEQ. | | | on | peptide | | histo- |
| | | ID | Starting | | protein | on rapid | Antibody | chemical |
| SARS virus protein | Peptide code | No. | position | Native peptide sequence | chip | test | code | study |
| Envelope/ | SARS-AgS18 | 1 | S60 | SRVKNLNSSEGVPDLLV | + | + | SARS-AbS18 | + |
| small envelope protein/ | | | | | | | | |
| sars 4 | | | | | | | | |
| Membrane/ | SARS-AgS17 | 2 | M1 | MADNGTITVEELKQLLEQ | + | + | SARS-AbS17 | + |
| PLPM/matrix/ | SARS-AgS16 | 3 | K204 | KLNTDHAGSNDNIALLVQ | + | + | SARS-AbS16 | + |
| sars 5 | SARS-AgS31 | 4 | N4 | NGTITVEELKQL | + | + | SARS-AbS31 | NT |
| Spike/ | SARS-AgS1 | 5 | D1135 | DKYFKNHTSPDVDLGD | NT | NT | SARS-AbS1 | + |
| spike glycoprotein/ | SARS-AgS2 | 28 | V1158 | VVNIQKEIDRLNEV | NT | NT | SARS-AbS2 | − |
| E2 glycoprotein- | SARS-AgS3 | 29 | Y899 | YENQKQIANQFNKA | − | − | SARS-AbS3 | − |
| precursor/ | SARS-AgS4 | 30 | S785 | SQILPDPLKPTKRSF | NT | NT | SARS-AbS4 | − |
| sars 2 | SARS-AgS5 | 6 | P540 | PSSKRFQPFQQFGRD | + | + | SARS-AbS5 | + |
| | SARS-AgS6 | 7 | K439 | KYRYLRHGKLRPFERD | NT | NT | SARS-AbS6 | + |
| | SARS-AgS7 | 8 | D204 | DVVRDLPSGFNTLKPI | − | − | SARS-AbS7 | + |
| | SARS-AgS8 | 9 | D24 | DVQAPNYTQHTSSMRG | − | − | SARS-AbS8 | + |
| | SARS-AgS23 | 10 | C1236 | CKFDEDDSEPVLKGVKLHYT | + | + | SARS-AbS23 | + |
| | SARS-AgS24 | 11 | C19 | CTTFDDVQAPNYTQHTSS | − | − | SARS-AbS24 | + |
| | SARS-AgS32 | 12 | G536 | GVLTPSSKRFQPFQQ | + | + | SARS-AbS32 | NT |
| | SARS-AgS33 | 13 | R553 | RDVSDFTDSVRDPKTSEI | + | + | SARS-AbS33 | NT |
| Nucleocapsid/ | SARS-AgS12 | 14 | R406 | RQLQNSMSGASADSTQA | + | + | SARS-AbS12 | + |
| PUP5/ | SARS-AgS13 | 15 | M1 | MSDNGPQSNQRSAPRIT | + | + | SARS-AbS13 | + |
| sars 9a | SARS-AgS25 | 31 | A13 | APRITFGGPTDSTDNNQN | − | − | SARS-AbS25 | NT |
| | SARS-AgS26 | 16 | T25 | TDNNQNGGRNGARPKQRRP | + | + | SARS-AbS26 | NT |
| | SARS-AgS27 | 17 | P38 | PKQRRPQGLPNNTASWFT | + | + | SARS-AbS27 | NT |
| | SARS-AgS28 | 18 | D341 | DDKDPQFKDNVILLNKHIDA | + | + | SARS-AbS28 | NT |
| | SARS-AgS29 | 19 | K356 | KHIDAYKTFPPTEPKKDKKK | + | + | SARS-AbS29 | NT |
| | SARS-AgS30 | 20 | K371 | KDKKKKTDEAQPLPQRQKKQ | + | + | SARS-AbS30 | NT |
| PUP1/ | SARS-AgS21 | 21 | A258 | CAMDPIYDEPTTTTSVPL | + | + | SARS-AbS21 | + |
| Sars 3a | SARS-AgS22 | 32 | K134 | KSKNPLLYDANYFVC | − | − | SARS-AbS22 | − |
| PUP2/ | SARS-AgS20 | 22 | K31 | KVTAFQHQNSKKTTK | − | − | SARS-AbS20 | + |
| Sars 3b | SARS-AgS19 | 23 | K135 | KHKKVSTNLCTHSFRKKQVR | + | + | SARS-AbS19 | + |
| PUP3/ | SARS-AgS15 | 24 | K45 | TKKNYSELDDEEPMELDYP | + | + | SARS-AbS15 | + |
| Sars 6 | | | | | | | | |
| PUP4/ | SARS-AgS14 | 33 | K85 | KLFIRQEEVQQELYSP | − | − | SARS-AbS14 | − |
| Sars 7a | SARS-AgS9 | 25 | C35 | CPSGTYEGNSPFHPLADN | − | − | SARS-AbS9 | + |
| PUP6/ | SARS-AgS11 | 26 | E28 | EDAMGQGQNSADPKVYP | + | + | SARS-AbS11 | + |
| Sars 9b | SARS-AgS10 | 27 | Q78 | QMTKLATTEELPDEF | − | − | SARS-AgS10 | + |

+: positive;
−: negative;
NT: not tested

Conjugated Peptides

In one embodiment of the invention, the peptide of the invention can be linked or conjugated to a carrier protein. Examples of carrier molecules include bovine serum albumin (BSA), keyhole limpet hemocyanin (KLH), tetanus toxoid, and the like. The carrier molecule can be selected from BSA and KLH. Conjugation may be carried out by methods known in the art. One such method is to combine a cysteine residue of the fragment with a thiol-reactive moiety on the carrier molecule such as a cysteine residue or a maleimide group.

SARS peptides, after conjugation to a carrier protein, can achieve the same specificity and sensitivity as that of unconugated SARS peptides. Therefore, these peptides are highly adaptive to the many different diagnostic platforms where molecular size is important. FIGS. 3, 4 and 5 show reactions of peptides conjugated to carrier proteins such as KLH and BSA with SARS sera, using Alexa 546 anti-human IgG as reporter.

The polypeptides of the present invention that can include an immunogenic or antigenic epitope can be fused to other polypeptide sequences. For example, the polypeptides of the present invention can be fused with the constant domain of immunoglobulins (IgA, IgE, IgG, IgM), or portions thereof (CH1, CH2, CH3, or any combination thereof and portions thereof) resulting in chimeric polypeptides. Such fusion proteins can facilitate purification and can increase half-life in vivo.

Antibodies

The inventors have also discovered antibodies that are immunoreactive to the peptides of the invention. In one embodiment, an antibody is described that can be reactive to a peptide with an amino acid sequence selected from SEQ ID Nos. 1-27. The antibody can also be reactive to a peptide comprising an amino acid sequence substantially homologous to a sequence from the group consisting of SEQ ID Nos. 1-27. In one embodiment, the antibody can be reactive to a peptide including an amino acid sequence at least 80% identical to a sequence selected from SEQ ID Nos. 1-27. In another embodiment, the antibody is reactive to a peptide including an amino acid sequence at least 85%, at least 90%, at least 95% or at least 98% or 99% identical to a sequence selected from SEQ ID Nos. 1-27. In another embodiment an antibody is provided that is reactive to a peptide including an amino acid sequence consisting essentially of at least one of SEQ ID Nos. 1-27. In another embodiment, the antibody is reactive to a peptide including an amino acid sequence consisting of at least one of SEQ ID Nos. 1-27.

The antibody can be, for example, a polyclonal antibody or a monoclonal antibody. The antibody can be produced in a host by injection of an immunoreactive peptide including an amino acid sequence selected from the group consisting of SEQ ID Nos. 1-27. In one embodiment, the antibody is an IgG antibody. The antibody can also be an IgM antibody. In one embodiment, the host is avian. The host can be a mammal. In one embodiment, the host can be a rabbit or a rodent. More typically the host is a human.

Typically an animal used for production of anti-antisera is a rabbit, a mouse, a rat, a hamster, a guinea pig, or a goat. Because of the relatively large blood volume of goats and rabbits, a goat or rabbit is sometimes a preferred choice for production of polyclonal antibodies. Antibodies, both polyclonal and monoclonal, specific for an epitope of the present invention may be prepared using conventional immunization techniques, as will be generally known to those of skill in the art. A composition including an epitope having a sequence represented in SEQ ID Nos. 1-27 can be used to immunize one or more experimental animals, such as a rabbit or mouse, which will then proceed to produce specific antibodies against the peptide epitope.

For polyclonal antisera, the peptides or antigens can be coupled to a carrier protein, such as KLH as described for peptides above. The KLH-peptide can be mixed with Freund's adjuvant and injected into guinea pigs, rats, goats or rabbits. Polyclonal antisera can be obtained, after allowing time for antibody generation, simply by bleeding the animal and preparing serum samples from the whole blood. Antibodies can be purified by any method, including by peptide antigen affinity chromatography.

Alternatively, monoclonal antibodies can be prepared using a SARS polypeptide (or immunogenic fragment or analog) and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In: Monoclonal Antibodies and T Cell Hybridomas, Elsevier, N.Y., 1981).

To obtain monoclonal antibodies, an experimental animal, such as a mouse, is injected with a composition including at least one peptide including a sequence selected from SEQ ID Nos. 1-27 or a combination thereof and a population of spleen or lymph cells is obtained. The spleen or lymph cells can then be fused with cell lines, such as human or mouse myeloma strains, to produce antibody-secreting hybridomas. Monoclonal antibodie clones to the desired peptide epitope or combination of epitopes are identified using standard techniques, such as ELISA and Western blotting.

In vitro methods are also suitable for preparing antibodies. Digestion of antibodies to produce fragments thereof, particularly, Fab′ fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, etc. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

In addition antibody fragments which contain specific binding sites for SARS peptides and antigens may be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse, W. D. et al (1989) Science 256:1275-1281).

The antibodies can be tested for specific recognition of a corresponding peptide by techniques known in the art, including by Western blot or immunoprecipitation analysis (described in Antibodies: A Laboratory Manual, (eds. E. Harlow and D. Lane, Cold Spring Harbor, N.Y., 1988)). The antibodies of the present invention may be monospecific, bispecific, trispecific or of greater multispecificity. Multispecific antibodies may be specific for different epitopes of a peptide of the present invention or may be specific for both a peptide of the present invention as well as for a heterologous epitope. Alternatively, an antibody can be conjugated to a second antibody to form an antibody heteroconjugate as described by Segal in U.S. Pat. No. 4,676,980, which is incorporated herein by reference in its entirety.

The antibodies of the invention typically can have a high affinity for the peptides of the invention. The antibodies are generally able to form a stable peptide-antibody complex with the peptides of the invention. The antibodies can also be reactive to peptides expressed in or on the SARS virus or in a sample from a host infected with a SARS-CoV. When contacted with an antigen that is not an isolated peptide of the invention, the antibodies can have the capacity to form a antigen-antibody complex with a peptide or protein to which they are immunogenic.

Antibodies can be attached or conjugated to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies of the present invention can be used in standard immunochemical procedures, such as ELISA and Western blot methods, as well as other procedures, such as immunohistology of tissues, that may utilize antibodies specific to the immunoreactive peptides of the invention such as immunoadsorbent protocols for purifying native or recombinant SARS-CoV proteins and peptides. The antibodies can be used as high affinity primary reagents for the identification of proteins immobilized onto a solid support matrix, such as nitrocellulose, polyacrylamide, nylon, or the like. In conjunction with gel electrophoresis and immunoprecipitation, the antibodies can be used as a single step reagent for use in detecting species-specific epitopes of SARS-CoV. Immunologically-based detection methods for use in conjunction with Western blotting include enzymatically-, radiolabel-, or fluorescently-tagged secondary antibodies against the primary antibody moiety are considered to be of particular use in this regard. Kits

The invention also provides kits for diagnosing a SARS-CoV infection in a host. The kits generally include a) at least one reagent that detects a SARS infection either by directly detecting a SARS virus peptide in a sample or by detecting host antibodies reactive with SARS virus derived peptides, and b) a measurable signal when the detecting agent detects a SARS infection. The measurable signal can be a reagent directly linked to the detecting agent, or in a container with the detecting agent, or a reagent in a separate container. The signal can be measurable visually, can require an additional means for reading the signal, and/or can require an additional reagent, such as a commercially available reagent.

a. Peptide kits

In one embodiment, a kit is provided that includes a) a peptide of the invention and b) a means for detecting formation of a peptide-antibody complex. The peptide of the diagnostic kit can typically bind to a reactive antibody in a sample and can form a peptide-antibody complex with an antibody that recognizes the peptide. In one embodiment, the kit further includes c) a signaling reagent which interacts with the peptide-antibody complex. Alternatively, the kit can include a signaling reagent that interacts with the peptide and not with the peptide-antibody complex. The kit can also include reagents for measuring a signal produced.

The peptide in the kit can be conjugated to another protein or peptide, or another molecule or to a solid substrate. The peptide can also be in a stabilized solution or lypholized and a kit can contain an appropriate solution for hydrolyzing a lyophilized peptide. The kit can also contain an appropriate solid medium for blotting the aforementioned peptide on. The peptides or conjugated peptides of the invention in the kit can also be linked to a solid substrate. The substrate can be, for example, a membrane, fiber filter or a glass substrate. Reaction can be carried out on, for example, an aldehyde treated glass substrate. The kit can also include peptides linked or conjugate to a nitrocellulose membrane. The peptides of the invention can also be conjugated to micro spheres.

Typically the peptide in the kit is in an array. The array is typically at least one peptide including an amino acid sequence of SEQ ID Nos. 1-27, and can be composed of a mixture of peptides of different sequences, including at least 2, 3, 4, 5, 6, 7, 8, 9, or 10 different sequence, or more than 10 or between 10 and 20 or 15 and 20, or up to 23 sequences, or can include additional peptides known in the art. The additional peptides can be peptides known for diagnosing viral infection or can be peptides known for other purposes. The array can also include antibodies of the invention. Protein and antibody combination arrays can be particularly useful for targeted diagnoses.

In one embodiment, the peptide reagent in the kit can be contained in one compartment. The means for detecting formation of a peptide-antibody complex can be contained in the same compartment or can be in an additional compartment. In some cases, the means for detection is an antibody to the peptide of the invention. The antibody can be linked to a molecule that provides a signal or the kit can include a further reagent that provides a signal upon binding to a antibody-peptide-peptide-antibody trimeric complex. The reagent antibody can be in the same compartment or in a separate compartment from the peptides of the invention.

Other optional components of the kit include, for example, agents to catalyze the synthesis measurable products, such as, for example, enzyme conjugate and enzyme substrate and chromogens, the appropriate buffers hybridization reactions, and instructions for carrying out the present method. The kit can contain for example, an appropriate reagent for detecting the presence of reactive antibodies to the peptide, such as an anti-human IgG antibody labeled with streptavidin-alkaline phosphatase. Furthermore, the kit can contain a detection agent such as nitroblue tetrazolium and 5-bromo-4-chloro-3-indlyl phosphate (BCIP). In these test kits, the detectable label can include an enzyme-linked antibody, a fluorescently tagged antibody, or a radiolabeled antibody. Typically, the kit comprises a labelled enzyme-linked antibody, and the kit further includes a third container means including a quantity of a substrate for the enzyme sufficient to produce a visually detectable product.

In addition, the kit can include a container which includes a positive control containing one or more antibodies reactive with the peptides of the invention and/or a container including a negative control without such antibodies.

In certain embodiments, the kit comprises a mixtures of peptides of the invention that provide a signal upon binding of antibodies in a host or in a sample and a means for detecting formation of a peptide-antibody complex. Mixtures of peptides can include a mixture of SEQ ID Nos. 2, 6, 10 and 20 or a mixture of SEQ ID Nos. 2, 6, 20 and 21, or SEQ ID Nos. 2 and 21, or SEQ ID Nos. 2 and 20. FIG. 7 shows a mixture of structural and hypothetical proteins in mixture 1 that consists of peptides of SEQ ID No. 2, KLH-bound SEQ ID No. 10, unconjugated SEQ ID No. 6 and KLH-bound SEQ ID No. 20, which reaches 92% sensitivity. A mixture of structural and hypothetical proteins in mixture 2 consists of peptides of KLH-bound SEQ ID No. 2, SEQ ID No. 6, KLH-bound SEQ ID No. 21 and KLH-bound SEQ ID No. 20, which also reaches 92% sensitivity. Typically, a means can be provided to differentiate between the formation of a single or multiple peptide-antibody complexes.

In certain embodiments, a reagent antibody is provided in the diagnostic kit that is reactive against the host or sample species and produces a measurable signal when contacted with an antibody of the host or sample species. The reagent antibody may be linked to an agent that produces a signal, such as a fluorescent molecule. In other embodiments, a second reagent antibody is provided that can bind to the reagent antibody and elicit a signal. In another embodiment, a reagent antibody is provided that is bound to the peptide of the invention in the kit and produces a measurable signal when displaced from the peptide.

The kit can typically be designed for conventional assay formats like ELISA, EIA, RIA, IRMA etc. The samples used can be collected from body fluids such as serum, blood, cerebrospinal fluid, tear fluid, nasopharyngeal aspirate, seamen, or sweat.

b. Antibody kits

The diagnostic kit of the invention can also include a) an antibody of the invention and b) a means for detecting formation of an antigen-antibody complex. The antibodies are typically capable of forming an antigen-antibody complex with a peptide or protein in, on, or derived from a SARS-CoV. In one embodiment, the kit further includes c) a signaling reagent which interacts with the antigen-antibody complex. Alternatively, the kit can include a signaling reagent that interacts with the experimental antibody of the invention and not with the antigen-antibody complex. The kit can also further include reagents for measuring a signal produced.

In one embodiment, a kit is provided that includes an antibody of the invention capable of binding to a peptide expressed by a SARS-CoV (an antigen) and a means for detecting formation of a antigen-antibody complex. The antibody can typically bind to an antigen in a sample to form an antigen-antibody complex. The kit can also include a peptide of the invention linked to an antibody of the invention. In one embodiment, the antibody and peptide are complexed in the kit and the kit comprises a means for measuring the disassociation of the peptide-antibody complex. In this embodiment, the peptide can include a signal producing agent such as a fluorescent marker. The kit can also include a separate container including an agent capable of eliciting a signal when contacting the peptide of the invention, which can be a separate antibody. The kit can also further include reagents for measuring a signal produced.

The antibody in the kit can be conjugated to another protein or peptide, or another molecule, or to a solid substrate. The antibody can also be in a stabilized solution or lypholized and a kit can contain an appropriate solution for hydrolyzing a lyophilized peptide. The kit can also contain an appropriate solid medium for blotting the aforementioned antibody on. The antibodies in the kit can also be linked to a solid substrate. The substrate can be, for example, a membrane, fiber filter or a glass substrate. Reaction can be carried out on, for example, an aldehyde treated glass substrate. The kit can also include peptides linked or conjugate to a nitrocellulose membrane. The peptides of the invention can also be conjugated to microspheres.

Typically the antibody in the kit is in an array. The array is typically at least one antibody reactive with a peptide of the invention, and can also comprise at least 2, 3, 4, 5, 6, 7, 8, 9, or 10 different antibodies, or more than 10 or between 10 and 20 or 15 and 20, or up to 23 antibodies. The antibody array can also include peptides known in the art. The additional peptides can be peptides known for diagnosing viral infection or can be peptides known for other purposes. The array can also include one or more peptides of the invention.

In one embodiment, the antibody in the kit is contained in one compartment. The means for detecting formation of a antigen-antibody complex can be contained in the same compartment or can be in an additional compartment. In some cases, the means for detection is a reagent antibody to the host species of the invention. The reagent antibody can be linked to a molecule that provides a signal or the kit can include a further reagent that provides a signal upon binding to a antibody-antigen-antibody trimeric complex. In another embodiment, a reagent antibody is provided that is bound to the peptide of the invention in the kit and produces a measurable signal when displaced from the peptide. The reagent antibody can be in the same compartment or in a separate compartment from the antibody of the invention.

The compartments in the kit can be solid substrates such as enclosed membranes or glass slides. The solid substrates can include means to interact so that only portions of the substrates contact. The compartments can be designed to include a reservoir for including a sample, such as a liquid sample. The kits can also further include buffers for diluting samples and tests for measuring sample suitability.

Other optional components of the kit include, for example, agents to catalyze the synthesis measurable products, such as, for example, enzyme conjugates and enzyme substrates and chromogens, the appropriate buffers hybridization reactions, and instructions for carrying out the method. The kit can contain for example, an appropriate reagent for detecting the presence of reactive antibodies to the peptide, such as an anti-human IgG antibody labeled with streptavidin-alkaline phosphatase. Furthermore, the kit can contain a detection agent such as nitoblue tetrazolium and 5-bromo-4-chloro-3-indlyl phosphate (BCIP). In these test kits, the detectably labeled antibody may be an enzyme-linked antibody, a fluorescently tagged antibody, or a radiolabeled antibody. Preferably, the detectably labeled antibody is an enzyme-linked antibody, and the kit further includes a third container means including a quantity of a substrate for the enzyme sufficient to produce a visually detectable product.

In addition, the kit can have a container which includes a positive control containing one or more peptides of the invention and/or a container including a negative control without such peptides. In certain embodiments, the kit comprises a mixtures of antibodies of the invention that provide a signal upon binding of an antigen from a host or in a sample and a means for detecting formation of a antigen-antibody complex.

The kit can be designed for conventional assay formats like ELISA, EIA, RIA, IRMA etc. The samples used are typically collected from body fluids such as serum, blood, cerebrospinal fluid, tear fluid, seamen or sweat.

Methods

The invention also provides methods of diagnosing the presence of SARS-CoV infection in a sample. The method can include detecting 1) viral antigens or 2) host immunoglobulin against these viral antigens or both 1) and 2). Diagnosis bases on antigen detection can be direct or indirect, typically a capture sandwich assay or competitive assay using pairs of specific antibodies. A detectable signal can be from a detection reporter system which can be an antibody against host IgG, IgM or IgA, a combination of any two or all of the three. In one embodiment, the detection reporter can be a direct label on one of the antibodies in a pair. The reporter can be linked to a secondary antibody against an IgG, IgM or IgA, or against a host if the antibody pairs are generated from different hosts, or a combination of any two, three, four or more antigens.

In one embodiment, the invention provides methods of diagnosing the presence of SARS-CoV infection in a sample comprising:

a. providing at least one peptide comprising at least one of SEQ ID Nos. 1-27;

b. contacting a sample with the peptide; and

c. detecting the presence of a peptide-antibody complex.

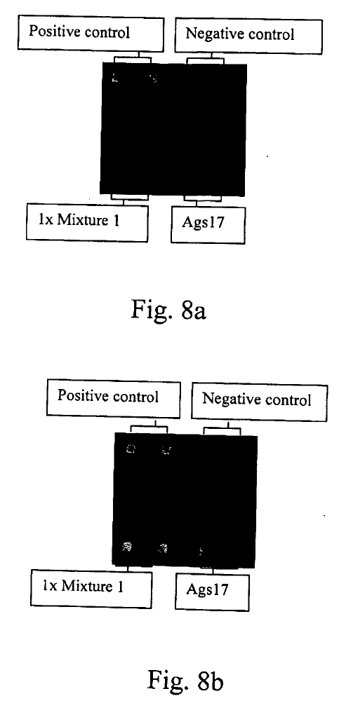

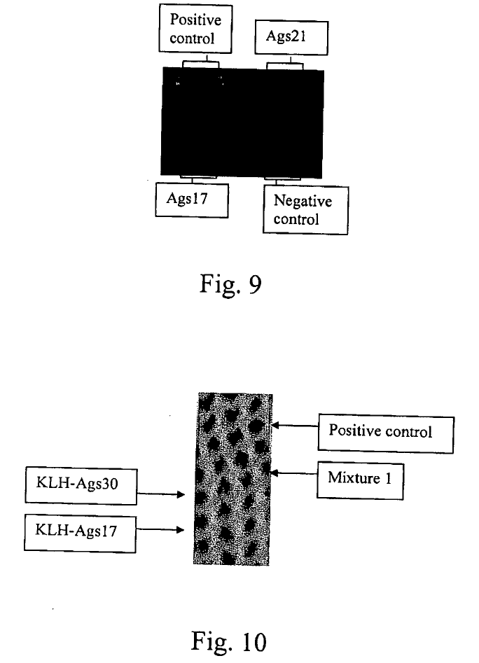

The peptide can be linked to a carrier molecule or to a solid support. FIG. 9, for example, shows the reaction of membrane protein Ags17 (SEQ ID No. 2) and hypothetical protein Ags21 (SEQ ID No. 21) printed on the on aldehyde treated glass substrate/slides (CSS; Telechem, USA) and reacted with a SARS serum sample using Alexa 546 anti-human IgG as the reporter. FIG. 10 shows the immunochromatographic reaction of SARS serum on nitrocellulose membrane, AE100 (S&S, USA). A mixture of peptides (mixture 1), Ags30 (SEQ ID No. 20) and Ags17 (SEQ ID No. 2) were printed on the membrane. Colloidal gold conjugated anti-human IgG was used as the reporter and therefore no visual aid or reader was required but merely an image taken. IgM can also be detected using the method described because the peptides of the invention react with IgG and IgM host antibodies. When anti-human IgM was used in the reporter system, a positive signal was detected on recently infected sera. FIG. 8 showed an IgG negative but IgM positive SARS case of a 7 days infected patient. Application of the dual reporter system of IgG and IgM, sensitivity of the SARS-CoV peptides for detection of SARS-CoV infection can typically approach 98%.

The method can also comprise a detection molecule or signaling agent that provides a measurable signal upon binding to the peptide-antibody complex. The method can also further include a secondary antibody that binds to the host species. In one embodiment, the secondary antibody is conjugated to a detection molecule or signaling agent that provides a measurable signal upon binding to the peptide-antibody complex.

One method of detecting SARS-CoV infection in a host generally includes the step of contacting a sample from the subject with an antibody having binding specificity for a peptide having an amino acid sequence selected from SEQ ID Nos. 1-27 under conditions allowing antigen-antibody binding to occur to form a complex and complex formation indicates the presence of SARS. In another embodiment, the invention provides methods of diagnosing the presence of SARS-CoV infection in a sample comprising:

a. providing at least one antibody capable of forming an antigen-antibody complex with a peptide comprising an amino acid sequence of at least one of SEQ ID Nos. 1-27 or with a peptide expressed by a SARS-CoV or a host infected with a SARS-CoV;

b. contacting a sample with the antibody; and

c. detecting the presence of an antigen-antibody complex.

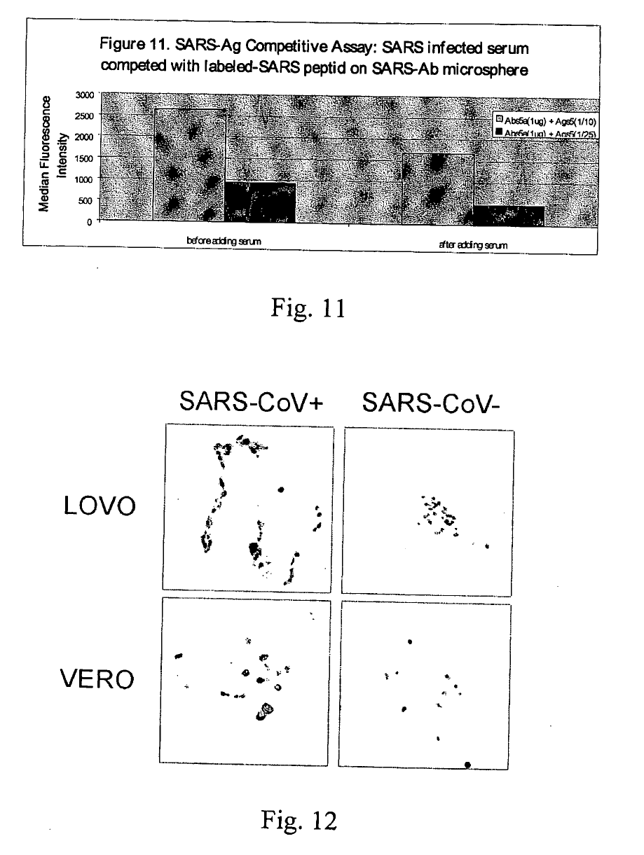

The method can further include contacting the complex with a reagent antibody that reacts with the antigen-antibody complex. The method can also include a peptide of the invention. In this embodiment, the method can include formation of a peptide-antibody complex before contact with the sample and measuring dissociation of the peptide-antibody complex to detect the antigen-antibody complex. In such a “antigen-competitive” assay, SARS infected serum was shown to be able to compete with labeled peptide to bind the antibodies immobilized on mcirosphere, and as a result, signal intensity dropped when compare to a normal control. FIG. 11 show a competitive assay using peptide SARS-Abs5 (SEQ ID No. 6) conjugated microsphere on a liquid chip platform. Approximately 50% of the original binding is competed off in the presence of infected serum.

The antibody can be conjugated to a carrier molecule or to a solid support. The solid support can be, for example, a well (typically in an assay plate) or a microsphere. In one embodiment, ‘spots’ of protein-antibody or antigen-antibody complex may form on the support. Quantifying the spots (and typically comparing against a control) can allow determination of recognition of a peptide- or antigen-antibody complex. After the sample is allowed to bind, the solid support can optionally be washed to remove material which is not specifically bound to the probe. The complex may be detected by using a second binding agent which will bind the complex. Typically the second agent binds the antibody or peptide at a site which is different from the site which binds the first probe. The second agent is typically an antibody and is labeled directly or indirectly by a detectable label. The second agent may also be detected by a third agent which is typically labeled directly or indirectly by a detectable label. For example the second agent may include a biotin moiety, allowing detection by a third agent which can include a streptavidin moiety and typically alkaline phosphatase as a detectable label.

The detection can be via any standard assay for detecting interaction between molecules. Assays that can be used include, but are not limited to, immunoprecipitation (Kang et al. (1997) Mol. Cells, 7:237-243; Gharbia et al. (1994) J. Peridontol. 65:56-61), immunohistology (Navarro et al., (1998) Neurosci. Lett. 254:17-20; Nitta et al. (1993) Biol. Reprod. 48:110-116; Heider and Schroeder, (1997) J. Virol. Methods, 66:311-316), immunoblotting (Beesley, J. E., Immunochemistry: A Practical Approach (IRL Press, Oxford, England, 1993), ELISA (Macri and Adeli (1993) B. Eur. J. Clin. Chem. Clin. Biochem. 31:441-446; Rodriguez et al. (1990) J. Dairy Res. 57:197-205), immunoelectrophoresis, immunofluorescence (Avarameas et al (1978) J. Immunol. 8, suppl. 7:7; Wilson and Nakane, Immunofluorescence and Related Staining Techniques, p215 (Elsevier/North Holland Biomedical Press Amsterdam, 1978), chromatography (for example, chromatography may use denaturing and/or non-denaturing conditions, and my involve, the use of any kind of resin, such as, Nickel Affinity, hydroxyapatite, silica, amino acids, carbohydrate binding matrices, carbohydrate matrices, chelating resins, ion exchange, anion exchange, HPLC, Liquid chromatography, immunoaffinity matrices and other specialized resins), western blotting, far western blotting, radioisotope labeling, luciferase assays, two-hybrid based assays (numerous two-hybrid based assays systems are commercially available), chemiluminescence assays and/or fluorescence assays. Other methods for detection of antigens and antibodies well known in a clinical laboratory setting are contemplated by the present invention, including: immunodiffusion, electrophoresis and immunoelectrophoresis, immunochemical and physicochemical methods, binder-ligand assays, immunohistochemical techniques (immunofluorescence), agglutination, IgG and IgM capture assay test, competitive inhibition assays for antibodies, or complement assays.

In one embodiment, an ELISA can be used to detect the formation of an antigen-antibody or peptide-antibody complex. In an ELISA assay, proteins or peptides incorporating species-specific sequences or species-specific combination of sequences are immobilized onto a selected surface, preferably a surface exhibiting a protein affinity such as the wells of a polystyrene microtiter plate. After binding of antigenic material to the well, coating with a non-reactive material to reduce background, and washing to remove unbound material, the immobilizing surface is contacted with a sample in a manner conducive to peptide- or antigen-antibody complex formation. Such conditions can include diluting the antisera with diluents such as BSA, bovine gamma globulin (EGG) and phosphate buffered saline (PBS)/Tween. The antisera is then allowed to incubate for from 0.5 to 48 hours, typically from 0.5-24 or from 0.5-10, 0.5-5, 0.5-4, 0.5-2, 1-2, 0.25, 0.5, 1 or 1.5 hours. Following incubation, the antisera-contacted surface is washed so as to remove non-immunocomplexed material.

The antibodies or peptides or detection agents may be labeled with radioisotopes (for example, ³², ³H, ¹³C and/or ¹²⁵I), Biotin Fluorescent molecules (for example,CY3, CY5, Fluorescein, DAPI, R-PPhycoerythrin, PKH2, PKH26, PKH67, Propidium Iodide, Quantum Red™, Rhodamine, Texas Red or others known in the art), Protein G, or A (which bind the Fc region of many mammalian IgG molecules) or protein L (which binds to the kappa light chains of various species), gold (for example, colloidal gold) and/or enzymes (preferably SARS peptides or antigens, where desirable and appropriate, are “tagged” with an epitope having available one or more antibodies or molecules which specifically bind (commercially available antibodies, specific to enzymes, molecules and epitope tags, are well known in the art)). A molecule having a “tag” (Pretorius et al. (1997) Onderstepoort J. Vet. Res. 64:201-203), includes, but not limited to, myc-, HA-, GST-, V-5-, Lex-A-, cI-, DIG-, Maltose binding protein-, Cellulose binding domain-, streptavidin, Alkaline phosphatase (O'Sullivan et al. (1978) FEBS Lett. 95:311-313), Horseradish Peroxidase, green fluorescent protein, 3×FLAG®-, HIS-Select™-, EZView™-S-Gal™-tags (available from Sigma, Life Science Research).

The occurrence and even amount of complex formation may be determined by subjecting the sample-contacted antibody or peptide to a second antibody having specificity for the a host species or an experimental antibody. To provide a detecting means, the second antibody will preferably have an associated enzyme that will generate a color development upon incubating with an appropriate chromogenic substrate. Thus, for example, one will desire to contact and incubate the antisera-bound surface with a urease or peroxidase-conjugated anti-appropriate-animal IgG for a period of time and under conditions which favor the development of immunocomplex formation (e.g., incubation for 2 hours at room temperature in a PBS-containing solution such as PBS-Tween).

The assay format can a singlet or multiplex assay. Assay platforms can be liquid or fluidic using coupled-beads and flow-cytometry technology or solid such as ELISA, tube rapid strip test or protein chip. The detectable label can be assayed by any desired means, including spectroscopic, photochemical, biochemical, immunochemical, radioisotopic, or chemical means. The probe can also be detected using techniques such as an oligomer restriction technique, a dot blot assay, a reverse dot blot assay, a line probe assay, and a 5′ nuclease assay. The length of time that the sample is in contact with the antibody or peptide of the invention can vary. Typically, this is less than two hours, and more often less than one hour. In certain cases it may be as short as one to ten minutes.

SARS is characterized by a non-specific onset and an incubation period of 2-10 days. A host can thus be tested over time. Typically, a host is tested over a period of one month or less after potential exposure, such as, for example, at any of days 1, 2, 3, 4, 5, 6, 7, 8, 9, 10, 11, 12, 15, 18, 21, 28 or 30. A host can also be tested at less than one day after potential exposure, or over long periods of time after exposure, such as to identify the epidemiological migration of the disease. A host can be tested, for example, at two months, six months, one year, or more than one year, such as at 2, 5, 10 or 20 years after potential exposure.

Vaccines

The invention also provides increasing a host immune response against SARS is provided comprising the peptides or antibodies of the invention. In one embodiment, a vaccine against SARS-CoV infection including a peptide or antibody of the invention is provided. A method of vaccinating a host is also provided including treating the host with a peptide or antibody of the invention. In another embodiment, a method of reducing SARS-CoV infection in a host including treating the host with an antibody of the invention is provided.

### EXAMPLES

### Example 1

### Peptide Development

Since the first emerge of SARS cases, various research groups successfully sequenced the SARS-CoV virus genome and assign names to hypothetical proteins differently. To avoid confusion, the naming scheme appeared in the sequence with GenBank accession number NC 004718 is adopted.

Computer assist analysis of the eleven reported SARS proteins, including spike glycoprotein (E2 glycoprotein precursor/S/Sars 2), matrix or membrane protein (M/Sars 5), nucleocapsid protein (N/Sars 9a), small membrane/envelope protein (E/Sars 4), and the hypothetical proteins Sars 1, Sars 3a/b, Sars 6, Sars 7a/b, Sars 8a/b and Sars 9b was accomplished by both public and commercial software. Relevant protein sequences of various SARS coronavirus isolates (Tor2, Urbani and BJ01) were extracted from public database of GenBank (Accession No: AY274119, AY278741 and AY278488). Multiple sequence alignment of the same protein from different isolates of SARS coronavirus was done to identify homology regions. Non-conserve regions were avoided in peptide design. Design of antigenic peptide in silico was aided by Clone Manager 5 (Scientific & Educational Software), Peptide Companion (CSPS Pharmaceuticals, Inc., San Diego, Calif.) and DS Gene (Accelrys). These peptides were also compared against other published coronaviruses such as human Group 1 and Group 2 coronaviruses, Bovine-CoV, Porcine-CoV, Feline-CoV and Canine-CoV to select peptides that were SARS coronavirus specific. Peptides that homologous with any of these sequences were not used.

15-20 amino acid residues of SARS proteins were synthesized by FMOC solid phase technique with an RAININ Symphony synthesizer. An additional cysteine (C) was added to either the C or N terminal of each peptide for further sulfhydryl conjugation. These peptides were purified by reverse phase C6 column. Peptide molecular size was confirmed by mass spec analysis. Peptides were synthesized by Abgent (San Diego, Calif.).

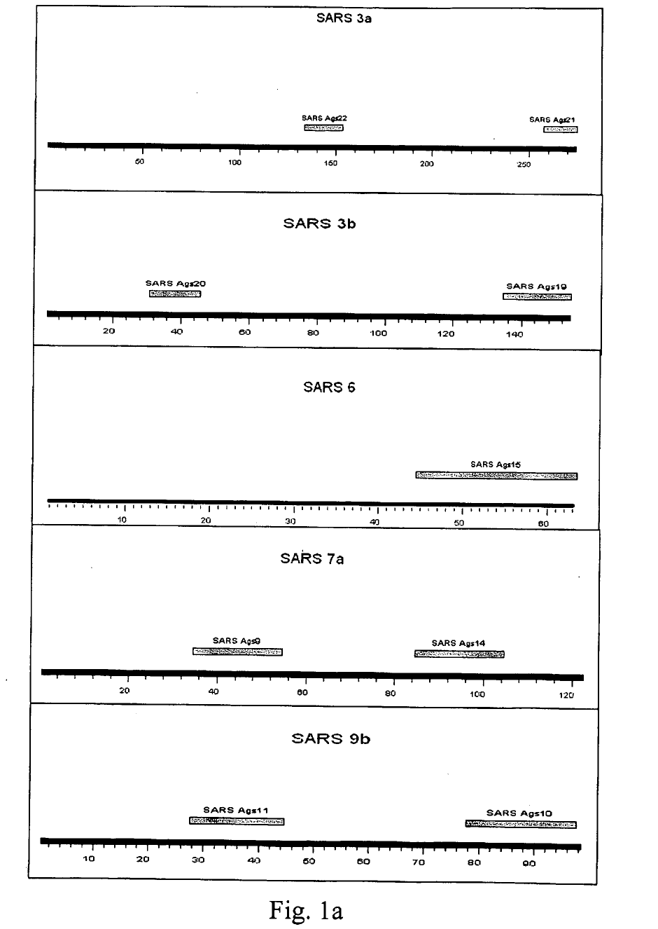

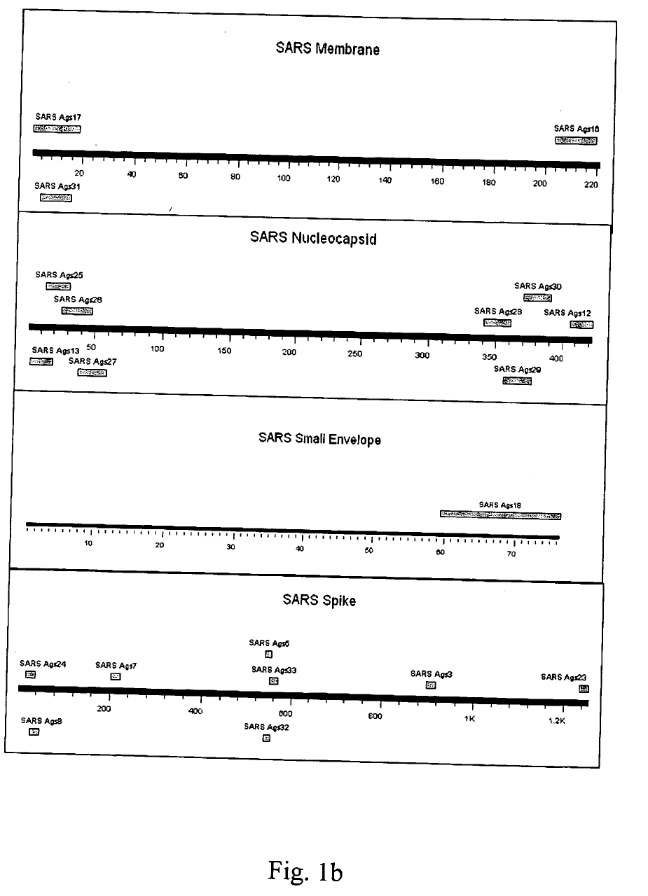

A total of 29 peptide fragments covering 9 of the proteins, structural and hypothetical, were synthesed. All the peptides and antibodies are screened by various immunological methods including protein chip, liquid chip, immunochromatographic strip test, immunohistochemistry. Immunoreactive reagents (peptides and antibodies) are marked by ‘+’ signs, while non-reactive reagents are marked by a ‘−’ signs. The sequence information and immunoreactivity test results are summarized in Table 1. Physical position of each peptide in relationship to the proteins found in SARS CoV genome is shown in FIG. 1.

### Example 2

### Antibody Production

Peptides of Example 1 were conjugated to carrier proteins such as bovine serum albumin (BSA) or keyhole limpet hemocyanin (KLH) employing the sulfhydryl-maleimide chemistry. Equal quantity of peptide and carrier protein was mixed to conjugate. Product was purified by gel filtration Protein G column.

Prior to immunization, pre-immune bleed is collected. 0.1 mg of each KLH conjugated SARS-peptide was used to immunized New Zealand rabbits subcutaneouly. Rabbits were boosted intramuscularly at one months intervals for 2 months, a total of three immune bleeds were collected. Serum from the immunized rabbits was tested in ELISA for reactivity and to determine the titre. IgG fraction in the serum was purified by protein G column.

### Example 3

### Diagnostic Protein Chip

Peptides or mixtures of peptides in phosphate buffered saline, either unconjugated or carrier protein conjugated, were spotted onto multichamber CSS slide and/or Fast Slide in an array of four by Cartesian Microsys fitted with Telechem SMP3B pin. After printing, the slides were baked and stored desiccated. Serum samples were mixed with 5% milk in PBS in a one to one ratio, added into the separate chambers and incubated at 37° C. for 1.5 hr while shaking. After aspirating the first reactants, the chambers were washed 3 times with washing buffer (0.1% Tween 20 in PBS). A secondary reagent (Alexa-546 labeled goat Fc anti-human IgG F(ab)2, or Alexa-647 labeled goat anti-human IgM or a mixture of the two secondary antibodies), was added to each chamber and incubated at 37° C. for 30 min. The chip was washed as described previously and fluorescence measured. Resulting fluorescence images were captured by ScanArray from Packard Bioscience (now known as Perkin Elmer). Image analysis was computer aided using QuantArray.

### Example 4

### Preparation Rapid Strip Test

Preparation of conjugation pad: Glass Fiber 33 or similar material was blocked with block reagents and then dried. Gold Conjugate pad was prepared by spaying goat anti-human IgG or goat anti-human IgM or a mixture of the two secondary antibodies, at a rate of 10 μL/cm using the AirJet Quanti on the blocked fiber and then dried at 37° C. for 1 hour.

Immobilization of test and control reagents on membrane: Peptides (test reagent), and rabbit anti-goat antibody (control reagent) were spaying separately on FF85 membrane or similar nitrocellulose membrane at a rate of 1 μL/cm to form the test (T) and control (C) lines respectively, using the BioJet Quanti devices on the BioDot XYZ 3000. The membrane was dried to immobilize the test and control reagents.

Assembly of lateral rapid strip test: All test strip assembly is performed in a humidity-controlled environment with relative humidity maintained at less than 20%. The sprayed membrane is placed on the adhesive face of a plastic backing. A strip of Wick 470 or any absorbent pad us overlayed towards the end downstream from the capture lines of the sprayed membrane. The membrane and pad overlap by 2 mm. The sprayed conjugate pad on the other end of the membrane with 2 mm overlapping at the margins of the membrane and the conjugate pad. A strip of CytoSep® or sample Pad is placed on the other end of the conjugate pad with 2 mm overlapping at the margins of sample pad and conjugate pad. At least one 4 mm wide test strip is cut and the test strip or strips are sealed in a light-proof foil pouch together with a desiccated pack.

Immunochromatographic reaction with serum samples: Undiluted or diluted serum samples e.g. 1/20 dilution in PBS buffer are used. Sample pad end of the strip is dipped into the sample vial and the sample migrates toward the other end for approximately 10 minutes. When both the Test- and Control-lines change from invisible to solid blue, the result is positive, when blue line appears only at the control-lines, the result is negative.

### Example 5

### Antigen Competitive Assay

To assay the immunoreactivity of SARS antibodies, competitive antigen assay was performed by Luminex liquid chip technology (Luminex Coperation, US). One microgram of SARS antibodies were coupled onto 5×10⁶ carboxylated-microsphere. The Antibody-micrcospheres were then allowed to bind to a specific ligand, i.e. biotin-conjugated peptide. The complex was then labeled by phycoerythrin-conjugated strepavidin. Median fluorescence intensity (MFI) on the peptide-antibody-microsphere complex was detected by Luminex X-100 system. The complex microspheres were then incubated with serum. The present of SARS antigen in serum competed with the labeled-peptides for binding to the antibody. Decrease in MFI was the measurable signal that detected the presence of SARS antigen.

### Example 6

### Immunohistochemical Studies of Anti-Peptide Antibodies of SARS-CoV on Autopsy and Biopsy Specimens

Antipeptide Antibodies

The antibodies were generated from rabbits immunized with a KLH-conjugated synthetic peptide selected from the N- or C-termini of the respective SARS-CoV proteins.

Antibodies and their concentrations and quantities tested in the immunohistochemical studies are listed in Table 2.

TABLE 2
Antibodies and their concentrations and quantities
tested in the immunohistochemical studies.

| | SARS antibodies | | |
|---|---|---|---|
| | General information | | |
| Protein | | concentration | |
| Group | GTHK code | (mg/ml) | Quantity (mg) |
| Envelope | SARS-AbS18 | 6.36 | 0.2 |
| Membrane | SARS-AbS17 | 7.36 | 0.2 |
| (PLPM) | SARS-AbS16 | 8.86 | 0.2 |
| Spike | SARS-AbS1 | 8.64 | 0.2 |
| | SARS-AbS2 | 14.36 | 0.2 |
| | SARS-AbS3 | 12.57 | 0.2 |
| | SARS-AbS4 | 13.92 | 0.2 |
| | SARS-AbS5 | 14.29 | 0.2 |
| | SARS-AbS6 | 14.43 | 0.2 |
| | SARS-AbS7 | 18.64 | 0.2 |
| | SARS-AbS8 | 20.36 | 0.2 |
| | SARS-AbS23 | 8 | 0.2 |
| | SARS-AbS24 | 7.64 | 0.2 |
| Nucleocapsid | SARS-AbS12 | 7.93 | 0.2 |
| | SARS-AbS13 | 6.36 | 0.2 |
| PUP1 | SARS-AbS21 | 8.14 | 0.2 |
| | SARS-AbS22 | 7.93 | 0.2 |
| PUP2 | SARS-AbS20 | 6 | 0.2 |
| | SARS-AbS19 | 6.21 | 0.2 |
| PUP3 | SARS-AbS15 | 6.71 | 0.2 |
| PUP4 | SARS-AbS14 | 5.64 | 0.2 |
| | SARS-AbS9 | 10.29 | 0.2 |
| PUP6 | SARS-AbS11 | 8.43 | 0.2 |
| | SARS-AbS10 | 7 | 0.2 |

Clinical Samples

Clinical specimens were selected retrospectively following the ethical guidelines of local ethical committee. The index patient of the Prince of Wales Hospital, Hong Kong, was a 26 year old man with a history of contact with the first SARS index case. Seven fatal SARS secondary cases, six of whom stayed in the same ward with the index patient and one with previous contact with the index patient while in the emergency department, were selected for the study. One probable SARS case (negative culture) during this same period of time and two additional lung sections of autopsies performed in early 2002 with diagnosis unrelated to lung pathology were used as negative controls. SARS was diagnosed according to the WHO criteria. Routine microbiological investigations, including serology, were performed on these patients with informed consent.

The biopsy was obtained during colonoscopy of one patient. Endoscopic appearance was noted and random biopsy specimens were taken from the colon and terminal ileum for histologic examination and viral detection. SARS-CoV infection in the terminal ileum was confirmed by RT-PCR and electron microscopy.

Cell Culture and SARS-CoV Infection

The cell lines Vero E6 (Monkey Kidney cell line) and LoVo (Human colon cancer cell line) were obtained from the American Type Culture Collection (ATCC, Manassas, Va.). The CUHK-W1 strain of SARS-CoV (GenBank accession No. AY278554) was grown in Vero cells and the third passage at a concentration of 5×10⁶ 50% tissue culture infective doses (TCID₅₀)/mL was kept at −70° C. for experiments. Cell lines at 60-70% confluence in 25-cm² flasks were inoculated with 300 μL of virus suspension to provide a multiplicity of infection (MOI) of 10. Inoculated cell cultures were incubated at 37° C. A mock infection was performed in parallel for each cell line. The cell monolayers were examined daily for cytopathic effects. Cells were harvested after seven days of incubation and SARS-CoV infection was confirmed by indirect immunofluorescence, in situ hybridiation and reverse transcriptase-polymerase chaing reaction (RT-PCR).

Immunohistochemical Studies

Cell blocks were prepared from 10% formalin-fixed trypsinized cells from infected or control Vero and LoVo cell cultures as in routine clinical cytology specimens. Four-micron sections were prepared from these cell blocks and from 10% formalin fixed, routinely processed paraffin blocks of biopsy or autopsy specimens. All available organs from autopsy were studied. Standard avidin-biotin method was used for immunohistochemical study using anti-peptide antibodies (1:100) with the StreptABComplex/HRP duet kit (DAKO, Glostrup, Denmark) according to the manufacturesr's protocols. Antigen retrieval was performed by microwave pretreatment in 10 mM citrate buffer, pH 6.0 or 0.1M EDTA buffer, pH 8.0, for preliminary heating at 780 W for 4 min, followed by 480 W for 5 min each for 2 times). Background of each antibody staining was evaluated in cell blocks prepared from non-infected Vero and LoVo cells.

Cell cultures, Vero E6 and LoVo, known to be permissive to SARS-CoV infection were used as positive controls and for the evaluation of the suitability of the antibodies in immunohistochemical studies on formalin-fixed paraffin-embedded sections. The results are summarized in Table 3. Representative results of SARS-Abs13a is shown in FIG. 12.

TABLE 3
Testing antipeptide antibodies on immunohistochemical
(ICH) study of human tissue samples from SARS-infected
patients, SARS-CoV infected Vero and LoVo cells.

| Protein Group | GTHK code | IHC | vero | lovo | background |
|---|---|---|---|---|---|
| Envelope | SARS-AbS18 | + | ++ | + | + |
| Membrane (PLPM) | SARS-AbS17 | − | + | − | − |
| | SARS-AbS16 | + | +++ | +++ | − |
| Spike | SARS-AbS1 | − | + | − | − |
| | SARS-AbS2 | − | − | − | − |
| | SARS-AbS3 | − | − | − | − |
| | SARS-AbS4 | − | − | − | − |
| | SARS-AbS5 | + | +++ | +++ | − |
| | SARS-AbS6 | − | + | − | − |
| | SARS-AbS7 | + | ++ | + | − |
| | SARS-AbS8 | − | + | +/− | + |
| | SARS-AbS23 | − | + | +/− | − |
| | SARS-AbS24 | + | ++ | ++ | − |
| Nucleocapsid | SARS-AbS12 | + | ++ | ++ | − |
| | SARS-AbS13 | + | +++ | +++ | − |
| PUP1 | SARS-AbS21 | + | +++ | +++ | − |
| | SARS-AbS22 | − | − | − | − |
| PUP2 | SARS-AbS20 | − | + | − | − |
| | SARS-AbS19 | − | ++ | − | − |
| PUP3 | SARS-AbS15 | − | + | − | + |
| PUP4 | SARS-AbS14 | − | − | − | − |
| | SARS-AbS9 | − | + | − | − |
| PUP6 | SARS-AbS11 | − | ++ | − | − |
| | SARS-AbS10 | − | ++ | − | − |

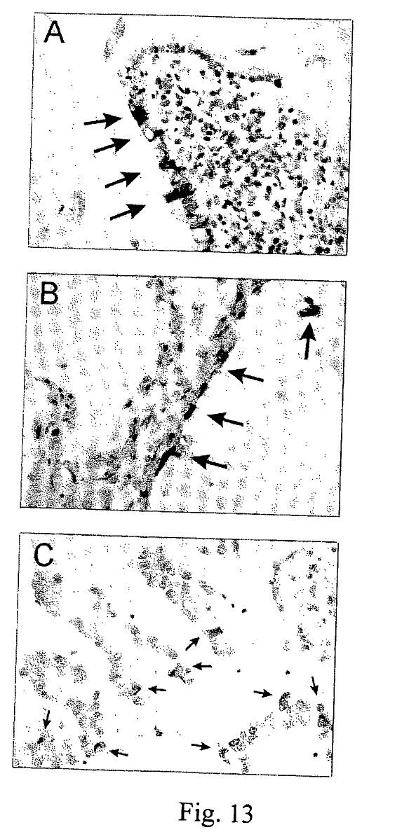

Antibodies that were found to work in fixed sections of infected culture cells were evaluated in biopsy and autopsy sections. All these antibodies preformed well in tissue sections. Result is summarized in Table 3, IHC column. In biopsy, both in situ hybridization and immunohistochemical analyses gave similar results. Only the surface enterocytes showed positive cytoplasmic signals. Crypt cells were rarely positive (FIG. 13A).

Results of immunohistochemical studies in biopsies from colonoscopy are listed in Table 4.

TABLE 4
Results of immunohistochemical studies
in biopsies from colonoscopy

| Biopsy | I | II |
|---|---|---|
| tissue | Terminal ileum | colon |
| Electron microscope | + | + |
| Immunohistochemical studies | + | − |
| In situ hybridization | + | − |
| (Probe: M gene) | | |

Autopsy specimens were degraded in most cases and were evaluated for suitability of immunohistochemical studies. Antipeptide antibodies were positive only in those sections where SARS-CoV was isolated and in situ hybridization positive. Representative sections of the lung and terminal ileum were shown in FIGS. 13B and C, respectively.

In both autopsy and biopsy cases, electron microscopy appeared to be the most sensitive test. Whereas SARS-CoV could not be isolated, or demonstrated by in situ hybridization or immunohistochemistry, only electron microscopy might be used to demonstrate the presence of viral particles.

What is claimed is:

1. An immunoreactive peptide comprising an amino acid sequence that is at least 80% identical to a peptide selected from the group consisting of SEQ ID NOs. 1-27.

2. The peptide according to claim 1 comprising an amino acid sequence selected from the group consisting of SEQ ID NOs. 1-27.

3. The peptide according to claim 1 consisting essentially of an amino acid sequence selected from the group consisting of SEQ ID NOs. 1-27.

4. The peptide according to claim 1 consisting of an amino acid sequence selected from the group consisting of SEQ ID NOs. 1-27.

5. The peptide of claim 1 further comprising a carrier molecule.

6. The peptide of claim 1 wherein the peptide is linked to a solid substrate.

7. An antibody immunologically reactive with the peptide according to claim 1.

8. The antibody of claim 7 wherein the antibody is a polyclonal antibody.

9. The antibody of claim 7 wherein the antibody is a monoclonal antibody.

10. The antibody of claim 7 wherein the antibody is an antibody fragment.

11. A diagnostic kit for the diagnosis of the SARS-CoV virus infection comprising the peptide according to claim 1, wherein said peptide is capable of forming a peptide-antibody complex in the presence of an immunoreactive antibody thereto.

12. The diagnostic kit according to claim 11, further comprising a signaling reagent that binds to said peptide-antibody complex.

13. The diagnostic kit according to claim 1 1, wherein said peptide is conjugated to a carrier molecule.

14. The diagnostic kit according to claim 11, wherein the kit includes an array of at least two peptides comprising an amino acid sequence at least 80% identical to a peptide selected from the group consisting of SEQ ID NO. 1-27.

15. A diagnostic kit for diagnosing the presence of the SARS-CoV virus or fragments thereof comprising an antibody immunological reactive with the SARS CoV virus and capable of forming an antigen-antibody complex therewith.

16. The diagnostic kit according to claim 15, further comprising a signally reagent capable of reacting with the antigen-antibody complex.

17. A diagnostic kit for diagnosing the presence of the SARS-CoV virus or fragments thereof comprising at least one antibody immunological reactive with the SARS CoV virus and capable of forming an antigen-antibody complex therewith and at least one peptide according to claim 1.

18. A method for the determination of a SARS-CoV virus infection comprising:
providing the peptide according to claim 1, said peptide capable of forming an antigen-antibody complex in the presence of a reactive antibody;
mixing a sample with said peptide; and
detecting the presence of an antigen-antibody complex.

19. The method according to claim 18 wherein said peptide is conjugated to a carrier molecule.

20. A method for the determination of the presence of a SARS-CoV in a test sample comprising:
providing an antibody immunologically reactive with the peptide of claim 1, said antibody capable of forming an antigen-antibody complex in the presence of the peptide, the SARS-CoV virus or a fragment thereof;
mixing a sample with said antibody; and
detecting the presence of the peptide-antibody complex.

21. A vaccine against the SARS-CoV infection comprising the peptide of claim 1.

22. A method for inducing an immune response, comprising administering a peptide comprising an amino acid sequence that is at least 80% identical to a peptide selected from the group consisting of SEQ ID NOs. 1-27.

23. A genetic vector comprising a nucleotide sequence selected from the group consisting of a nucleotide sequence encoding a peptide comprising one of SEQ ID NOs. 1-27, a peptide consisting essentially of one of SEQ ID NOs. 1-27, and a peptide consisting of one of SEQ ID NOs. 1-27.

24. A genetic vector comprising a nucleotide sequence encoding a peptide having an amino acid sequence that is at least 80% identical to a peptide selected from the group consisting of a peptide comprising one of SEQ ID NOs. 1-27, consisting essentially of SEQ ID NOs. 1-27, and a peptide consisting of one of SEQ ID NOs. 1-27.

25. A micro-organism having a genetic composition comprising a nucleic acid sequence encoding a peptide, said peptide having an amino acid sequence that is at least 80% identical to a peptide selected from the group consisting of a peptide comprising one of SEQ ID NOs. 1-27, a peptide consisting essentially of one of SEQ ID NOs. 1-27, and a peptide consisting of one of SEQ ID NOs. 1-27.


SEQUENCE LISTING (33 sequences)

SEQ ID NO: 1 - protein, 17 aa, Artificial Sequence
  Synthetic peptide sequence
SRVKNLNSSEGVPDLLV

SEQ ID NO: 2 - protein, 18 aa, Artificial Sequence
  Synthetic peptide sequence
MADNGTITVEELKQLLEQ

SEQ ID NO: 3 - protein, 18 aa, Artificial Sequence
  Synthetic peptide sequence
KLNTDHAGSNDNIALLVQ

SEQ ID NO: 4 - protein, 12 aa, Artificial Sequence
  Synthetic peptide sequence
NGTITVEELKQL

SEQ ID NO: 5 - protein, 16 aa, Artificial Sequence
  Synthetic peptide sequence
DKYFKNHTSPDVDLGD

SEQ ID NO: 6 - protein, 15 aa, Artificial Sequence
  Synthetic peptide sequence
PSSKRFQPFQQFGRD

SEQ ID NO: 7 - protein, 16 aa, Artificial Sequence
  Synthetic peptide sequence
KYRYLRHGKLRPFERD

SEQ ID NO: 8 - protein, 16 aa, Artificial Sequence
  Synthetic peptide sequence
DVVRDLPSGFNTLKPI

SEQ ID NO: 9 - protein, 16 aa, Artificial Sequence
  Synthetic peptide sequence
DVQAPNYTQHTSSMRG

SEQ ID NO: 10 - protein, 20 aa, Artificial Sequence
  Synthetic peptide sequence
CKFDEDDSEPVLKGVKLHYT

SEQ ID NO: 11 - protein, 18 aa, Artificial Sequence
  Synthetic peptide sequence
CTTFDDVQAPNYTQHTSS

SEQ ID NO: 12 - protein, 15 aa, Artificial Sequence
  Synthetic peptide sequence
GVLTPSSKRFQPFQQ

SEQ ID NO: 13 - protein, 18 aa, Artificial Sequence
  Synthetic peptide sequence
RDVSDFTDSVRDPKTSEI

SEQ ID NO: 14 - protein, 17 aa, Artificial Sequence
  Synthetic peptide sequence
RQLQNSMSGASADSTQA

SEQ ID NO: 15 - protein, 17 aa, Artificial Sequence
  Synthetic peptide sequence
MSDNGPQSNQRSAPRIT

SEQ ID NO: 16 - protein, 19 aa, Artificial Sequence
  Synthetic peptide sequence
TDNNQNGGRNGARPKQRRP

SEQ ID NO: 17 - protein, 18 aa, Artificial Sequence
  Synthetic peptide sequence
PKQRRPQGLPNNTASWFT

SEQ ID NO: 18 - protein, 20 aa, Artificial Sequence
  Synthetic peptide sequence
DDKDPQFKDNVILLNKHIDA

SEQ ID NO: 19 - protein, 20 aa, Artificial Sequence
  Synthetic peptide sequence
KHIDAYKTFPPTEPKKDKKK

SEQ ID NO: 20 - protein, 20 aa, Artificial Sequence
  Synthetic peptide sequence
KDKKKKTDEAQPLPQRQKKQ

SEQ ID NO: 21 - protein, 18 aa, Artificial Sequence
  Synthetic peptide sequence
CAMDPIYDEPTTTTSVPL

SEQ ID NO: 22 - protein, 15 aa, Artificial Sequence
  Synthetic peptide sequence
KVTAFQHQNSKKTTK

SEQ ID NO: 23 - protein, 20 aa, Artificial Sequence
  Synthetic peptide sequence
KHKKVSTNLCTHSFRKKQVR

SEQ ID NO: 24 - protein, 19 aa, Artificial Sequence
  Synthetic peptide sequence
TKKNYSELDDEEPMELDYP

SEQ ID NO: 25 - protein, 18 aa, Artificial Sequence
  Synthetic peptide sequence
CPSGTYEGNSPFHPLADN

SEQ ID NO: 26 - protein, 17 aa, Artificial Sequence
  Synthetic peptide sequence
EDAMGQGQNSADPKVYP

SEQ ID NO: 27 - protein, 15 aa, Artificial Sequence
  Synthetic peptide sequence
QMTKLATTEELPDEF

SEQ ID NO: 28 - protein, 14 aa, Artificial Sequence
  Synthetic peptide sequence
VVNIQKEIDRLNEV

SEQ ID NO: 29 - protein, 14 aa, Artificial Sequence
  Synthetic peptide sequence
YENQKQIANQFNKA

SEQ ID NO: 30 - protein, 15 aa, Artificial Sequence
  Synthetic peptide sequence
SQILPDPLKPTKRSF

SEQ ID NO: 31 - protein, 18 aa, Artificial Sequence
  Synthetic peptide sequence
APRITFGGPTDSTDNNQN

SEQ ID NO: 32 - protein, 15 aa, Artificial Sequence
  Synthetic peptide sequence
KSKNPLLYDANYFVC

SEQ ID NO: 33 - protein, 16 aa, Artificial Sequence
  Synthetic peptide sequence
KLFIRQEEVQQELYSP